(19) United States
(12) Patent Application Publication
(10) Pub. No.: US 2026/0274794 A1
(43) Pub. Date: Sep. 17, 2026

(54) COMPOUNDS, COMPOSITIONS, AND METHOD OF INHIBITING TAU PROTEIN AND ALPHASYNUCLEIN AGGREGATION

(71) Applicant: Purdue Research Foundation, West Lafayette, IN (US)

(72) Inventor: Jessica Sonia FORTIN, West Lafayette, IN (US)

(21) Appl. No.: 19/168,717

(22) PCT Filed: Mar. 22, 2024
(86) PCT No.: PCT/US2024/021106

§ 371 Date: Sep. 24, 2025

Related U.S. Application Data
(60) Provisional application No. 63/454,352, filed on Mar. 24, 2023.
(60) Provisional application No. 63/461,369, filed on Apr. 24, 2023.

(51) Int. Cl. C07D 209/08; A61K 31/404; A61P 25/28; C07D 409/12

(52) U.S. Cl. CPC C07D 209/08; A61K 31/404; A61P 25/28; C07D 409/12

(57) ABSTRACT

Compounds comprising aminoindole carboxamide, compositions comprising same, and method of using such compounds and compositions to inhibit tubulin-associated unit (tau) protein aggregation or alpha-synuclein (a-syn) protein aggregation in a subject having, or at risk for, tau protein aggregation or a-syn protein aggregation, respectively.

### CROSS-REFERENCE TO RELATED APPLICATIONS

This application claims the benefit of U.S. Provisional Application No. 63/454,352, filed Mar. 24, 2023, and U.S. Provisional Application No. 63/461,369, filed Apr. 24, 2023, each of which is incorporated by reference as if fully set forth herein.

### STATEMENT OF GOVERNMENT SUPPORT

This invention was made with government support under contracts AG070447 and AG071985 awarded by the National Institutes of Health. The government has certain rights in the invention.

### TECHNICAL FIELD

This disclosure relates to compounds comprising aminoindole carboxamide, compositions comprising same, and the use of such compounds and compositions to inhibit tubulin-associated unit (tau) protein and alpha-synuclein (α-syn) protein aggregation, including, but not limited to, neurofibrillary tangles (NFTs), associated with tauopathies (e.g., Alzheimer's disease, Downs syndrome, progressive supranuclear palsy, and traumatic brain injury) and Lewy bodies, associated with synucleinopathies (e.g., Parkinson's disease, dementia with Lewy bodies (DLB), and multiple system atrophy (MSA)).

### BACKGROUND

Neurodegenerative diseases such as Alzheimer's (AD) and Parkinson's (PD) can trace their origin to protein fibrillization, the process by which a misfolded protein develops fibrils. Two major neuropathological hallmarks characterize typical AD, the accumulation of extracellular β-amyloid (Aβ) plaques and neurofibrillary tangles (NFTs) in the brain. NFTs contain misfolded and hyperphosphorylated tubulin-associated unit (tau) protein. Hypothetically, Aβ plaques accumulate in the brain and consequently activate microglia-neuroimmune cells involved in sensing, housekeeping, and defense of the central nervous system (CNS). These microglial cells then induce a pre-inflammatory state, which contributes to the hyperphosphorylation of tau (p-tau). The insolubility of tau protein fibrils leads to NFTs, which result in neuron toxicity and an acceleration of AD neurodegeneration. The spatiotemporal distribution of NFTs has been shown to coincide with neurodegeneration and cognitive impairment.

There are six naturally occurring isoforms of the tau protein in the human brain. The isoforms vary by the presence or absence of inserts encoded by the Tau gene. The six isoforms (0N3R, 0N4R, 1N3R, 1N4R, 2N3R, 2N4R) are transcribed in the human brain by alternative splicing and are expressed in different ratios during neurodegeneration resulting in tau pathological effects. Each isoform is distinguished by two criteria: the absence, presence, or partial presence of two 29-amino acid inserts encoded by exons 2 and 3 resulting in 0N (no insert), 1N (one insert), or 2N (two inserts) tai; and the presence (4R) or absence (3R) of the R2 domain of the microtubule binding domain region (MTBR) in addition to the already present R1, R3, and R4 domains. Tau isoforms are differentially expressed across the hippocampus during various stages of development; in fetal stages only the 0N3R isoform is expressed, while all six isoforms are expressed in adulthood. In healthy adults the ratio of 3R to 4R isoforms is approximately 1 In AD brains, this ratio becomes skewed with the 3R isoform becoming preferentially added to the tau fibrils as the disease progresses. Isoform 4R on the other hand is more efficient at promoting microtubule assembly compared to 3R, with the R1-R2 region being unique to 4R-Tau; when the ratio of 4R to 3R drastically increases in the human brain this leads to Argyrophilic Grain Disease (AGD), a form of dementia. The 1:1 ratio of 3R and 4R isoforms is important to maintain as seen by the progression of neurodegenerative diseases when this ratio is altered towards the 3R or 4R direction.

An antibody, which apparently stimulates the clearance of amyloid plaques has been approved by the Food and Drug Administration for the treatment of AD. However, it is the formation of the neurofibrillary tangles and the spatiotemporal distribution of the tangles that correlate with the loss of cognition. In view of the foregoing, there is an unmet need for a small molecule that can inhibit the formation of oligomers, i.e., early-stage aggregation. Accordingly, it is an object of the present disclosure to provide such small molecules and related compositions, which can be used to inhibit tau protein aggregation and alpha-synuclein (α-syn) protein aggregation (e.g., Lewy bodies and Lewy neurites). This and other objects, as well as inventive features, will be apparent from the detailed description provided herein.

### SUMMARY

Provided is a compound of the formula:

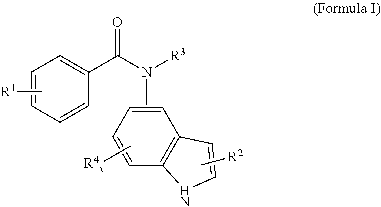

or a pharmaceutically acceptable salt thereof,
wherein:
R¹ is H, R^{a}O—, N≡C—, NO₂, NH₂, X₃C— or X, R^{a} is C₁-C₆ alkyl, and X is a halo;
R² is C₁-C₆ alkyl or X, wherein X is a halo;
R³ is H, C₁-C₆ alkyl or X, wherein X is a halo; and
R⁴ₓ in which x is 1, 2 or 3, and each R⁴ is independently H, C₁-C₆ alkyl or X, wherein X is a halo. In the compound of formula I, X can be Cl, F, I or Br. The compound of formula I can have the structure:

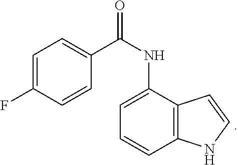 [SMILES: O=C(Nc1cccc2[nH]ccc12)c1ccc(F)cc1]

Also provided is a compound of the formula:

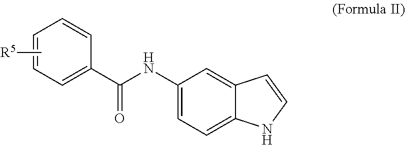

or a pharmaceutically acceptable salt thereof,
wherein:
R⁵ is H, R^{b}O—, N≡C—, NO₂, NH₂, X₃C— or X, R^{b} is a C₁-C₆ alkyl, and X is a halo. In the compound of formula II, X can be Cl, F, I or Br.

Also provided are compounds of the formula:

A compound of the formula:

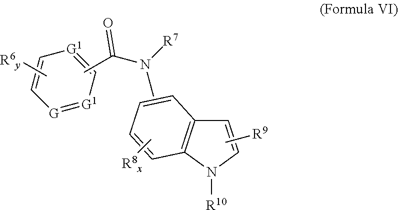

or a pharmaceutically acceptable salt thereof,
wherein:
G is N, CR⁶ or absent;
each G¹ is independently N, S, CR;
each R⁶ is independently H, alkyl, alkoxy, aryl, aryloxy, alkoxy, N≡C—, NO₂, amino, X₃C— or halo or two R⁶ form a cycloalkyl group, an arylcycloalkyl group, an aryl group or a heterocyclyl group;
y is 1, 2, 3, or 4;
R⁷ is H or C₁-C₆ alkyl;
each R⁸ is C₁-C₆ alkyl or X, wherein X is a halo;
x is 1, 2 or 3; and
R⁹ is H, C₁-C₆ alkyl or X, wherein X is a halo.

Further provided is a pharmaceutical composition comprising the compound of formula I or VI and a pharmaceutically acceptable carrier. In the compound of formula I, X can be Cl, F, I or Br. The compound of formula I can have the structure:

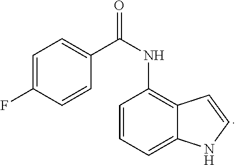 [SMILES: O=C(Nc1cccc2[nH]ccc12)c1ccc(F)cc1]

Still further provided is a pharmaceutical composition comprising the compound of formula II and a pharmaceutically acceptable carrier. In the compound of formula II, X can be Cl, F, I or Br.

Even still further provided is a method of inhibiting tubulin-associated unit (tau) protein aggregation in a subject having, or at risk for, tau protein aggregation. The method comprises administering to the subject an above-described pharmaceutical composition in an amount effective to inhibit tau protein aggregation. The subject can have, or be at risk for, Alzheimer's disease.

A method of inhibiting alpha-synuclein (α-syn) protein aggregation in a subject having, or at risk for, α-syn protein aggregation. The method comprises administering to the subject an above-described pharmaceutical composition in an amount effective to inhibit α-syn protein aggregation. The subject can have, or be at risk for, Parkinson's disease.

### BRIEF DESCRIPTION OF THE FIGURES

FIG. 1 shows reagents and conditions for the preparation of amides. 4-(dimethylamino) pyridine (4-DMAP), 1-ethyl-3-(3′-dimethyl aminopropyl) carbodiimide hydrochloride (EDCI·HCl), anhydrous dimethylformamide (DMFW), 0° C. to room temperature, 12 hours.

FIG. 2 shows kinetics of n-synuclein (r-syn) fibril formation for 18 carboxamide compounds compared to control (CTRL; DMSO at 0.25% (v/v)) as monitored using thioflavin T (ThT) fluorescence assays.

FIG. 3 shows kinetics of tau isoform 2N4R fibril formation obtained with pre-selected carboxamide derivatives compared to control (CTRL; DMSO at 0.25% (v/v)) as monitored using ThT fluorescence assays.

FIGS. 4A-4B are dose response curves showing the reduction of protein fibrillation for α-syn at approximately 83 hours (FIG. 4A) and tau isoform 2N4R at 50 hours (FIG. 43) based on the concentration of the three most potent compounds (2, 8 and 17) as monitored by ThT fluorescence assay.

FIG. 5 shows transmission electron micrographs of the reduction of α-syn (top row) and tau 2N4R fibril (bottom row) formation by compounds 2, 8 and 17 compared to control (CTRL; 0.25% DMSO). Scale bars located at the lower right corners are representative of 200 nm.

FIGS. 6A-6B show inclusion integrated intensity (fold change) (FIG. 6A) and confluence (fold change (FIG. 6B). Data are presented as fold-change relative to 0.1% DMSO. Ordinary one-way ANOVA plus Dunnett's multiple comparison test (*, p<0.1;***, p<0.001; **, p<0.0001).

### DETAILED DESCRIPTION

The present disclosure is predicated, at least in part, on the discovery that compounds comprising 4-aminoindole carboxamide can inhibit the aggregation of tubulin-associated unit (tau) protein, in particular the trimeric 2N4R isoform, which is the largest isoform at 67 KDa and more toxic than the trimeric 1N4R isoform in nondifferentiated neurons. Extracellular phosphorylated tau isoform 2N4R p-tau induces microglia proliferation and loss of neurons at low concentrations while inducing necrosis of the neurons and microglia at high concentrations, with its neurotoxicity being dependent on its aggregation activity. Due to its cytotoxic nature, the 2N4R isoform of tau is an excellent target for screening small molecules that could potentially be used to reduce NFT aggregation. Compounds comprising 5-aminoindole carboxamide are also effective but may not be as potent as compounds comprising 4-aminoindole carboxamide.

In view of the foregoing, provided is a compound of the formula:

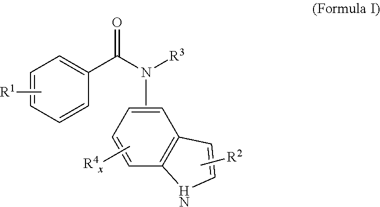

or a pharmaceutically acceptable salt thereof
wherein:
R¹ is H, R^{a}O—, N≡C—, NO₂, N₂, X₃C— or X, R^{a} is C₁-C₆ alkyl, and X is a halo;
R² is C₁-C₆ alkyl or X, wherein X is a halo;
R³ is H, C₁-C₆ alkyl or X, wherein X is a halo; and
R⁴, in which x is I, 2 or 3, each R₄ is independently H, C₁-C₆ alkyl or X, wherein X is a halo. In the compound of formula I, X can be Cl, F, I or Br. The compound of formula I can have the structure:

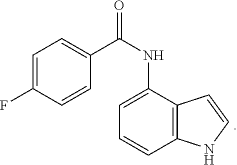 [SMILES: O=C(Nc1cccc2[nH]ccc12)c1ccc(F)cc1]

Also provided is a compound of the formula:

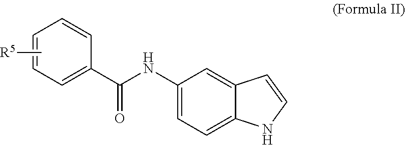

or a pharmaceutically acceptable salt thereof,
wherein:
R⁵ is H, R^{b}O—, N≡C—, NO₂, NH₂, X₃C— or X, R^{b} is a C₁-C₆ alkyl, and X is a halo. In the compound of formula II, X can be Cl, F, I or Br.

Also provided is a compound of the formula:

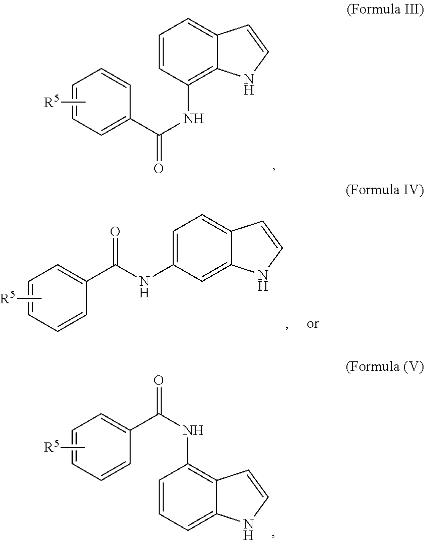

or a pharmaceutically acceptable salt thereof,
wherein:
R⁵ is H, R^{b}O—, N≡C—, NO₂, NH₂, X₃C— or X, RY is a C₁-C₆ alkyl, and X is a halo. In the compound of formula II, X can be Cl, F, I or Br.

Also provided is a compound of the formula:

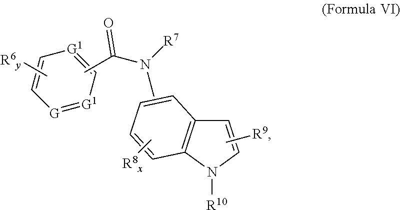

such as:

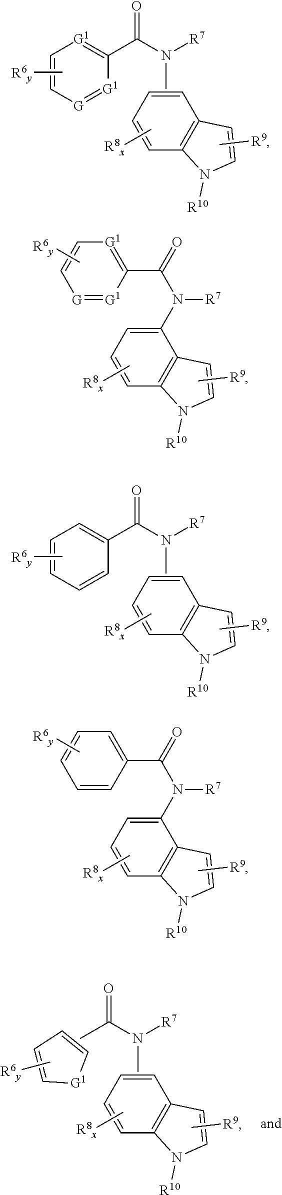

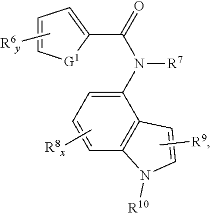

or a pharmaceutically acceptable salt thereof,
wherein:
G is N, CR⁶ or absent;
each G is independently N, S. or CR⁶;
each R⁶ is independently H, alkyl, alkoxy, aryl, aryloxy, alkoxy, N≡C—, NO₂, amino, X₃C— or halo or two R⁶ form a cycloalkyl group, an arylcycloalkyl group, an aryl group or a heterocyclyl group;
y is 1, 2, 3, or 4;
R⁷ is H or C₁-C₆ alkyl;
each R⁸ is C₁-C₆ alkyl or X, wherein X is a halo;
x is 1, 2 or 3; and
R⁹ is H, C₁-C₆ alkyl or X, wherein X is a halo.

The group

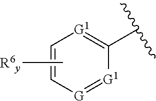

can be, for example, a group of the formula:

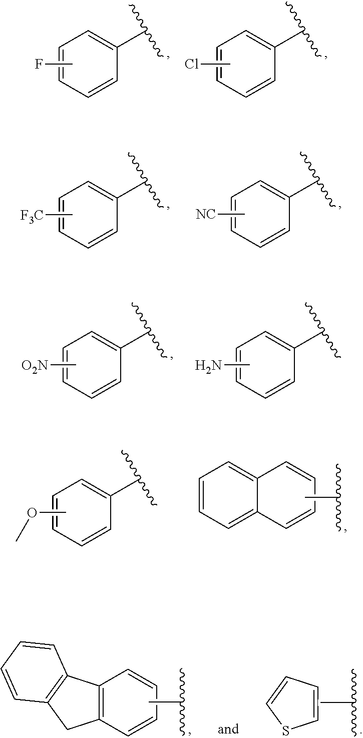

In the compounds of the formula VI, each G¹ can be CR⁶. Alternatively, or in addition, G can be CR⁶. Alternatively, or in addition, R⁷ and/or R¹⁰ can be C₁-C₃ alkyl. Alternatively, or in addition, y can be 2 or 3, and R⁶ can be halo. Alternatively, or in addition, y can be 1, and R⁸ can be H. Alternatively, or in addition, R⁹ can be H.

The above compounds include isotopic variants and compounds in which one or more hydrogen atoms have been substituted with deuterium. The above compounds also include stereoisomers.

The above compounds, and pharmaceutically acceptable salts and solvates, such as hydrates, thereof, can be synthesized in accordance with methods known in the art and exemplified herein. See, e.g., Example 1, which describes a method that can be utilized for 4-, 5-, 6- and 7-aminoindoles.

The compounds can be used to inhibit tau protein aggregation in tauopathies. Tauopathies are a group of disorders that result from abnormal tau phosphorylation, abnormal levels of tau, abnormal tau splicing, and mutations in the tau gene, for example. Neurodegenerative diseases have been classified based on this protein accumulation. Tauopathies encompass more than 20 clinicopathological conditions, including Alzheimer's disease (AD), which is the most common tauopathy. Other tauopathies include, but are not limited to, familial AD, primary age-related tauopathy (PART), Creutzfeldt-Jacob disease, dementia pugilistica, Gerstmann-Straussler-Scheinker disease (GSS), inclusion-body myositis, cortico-basal degeneration (CBD), Picks disease (PiD), progressive supranuclear palsy (also known as Steele, Richardson, and Olszewski disorder), Down syndrome, Parkinsonism with dementia, myotonic dystrophy, prion protein cerebral amyloid angiopathy, traumatic brain injury (TBI), amyotrophic lateral sclerosis (ALS), Parkinsonism-dementia complex of Guam, non-Guamanian motor neuron disease with neurofibrillary tangles, argyrophilic grain disease, diffuse neurofibrillary tangles with calcification, frontotemporal dementia with Parkinsonism linked to chromosome 17 (FTDP-17), Haller-vorden-Spatz disease, multiple system atrophy (MSA), Niemann-Pick disease type C, pallido-ponto-nigral degeneration, progressive subcortical gliosis, progressive supranuclear palsy (PSP), subacute sclerosing panencephalitis, tangle predominant dementia, postencephalitic Parkinsonism, myotonic dystrophy, subacute sclerosis panencephalopathy, mutations in LRRK2, chronic traumatic encephalopathy (CTE), familial British dementia, familial Danish dementia, other frontotemporal lobar degenerations, Guadeloupean Parkinsonism, neurodegeneration with brain iron accumulation, SLC9A6-related mental retardation, white matter tauopathy with globular glial inclusions, epilepsy, Lewy body dementia (LBD), mild cognitive impairment (MCI), multiple sclerosis, Parkinson's disease, HIV-related dementia, adult onset diabetes, senile cardiac amyloidosis, glaucoma, ischemic stroke, psychosis in AD, Huntington's disease, and prion diseases with tangles. The majority of neurodegenerative diseases are characterized by the deposition of insoluble protein in cells of the neuromuscular system.

The compounds can be formulated as pharmaceutical compositions comprising a pharmaceutically acceptable carrier using methods well-known in the art. “Carrier” is used generically herein to refer to pharmaceutically acceptable carriers, diluents, adjuvants, and excipients. See, e.g., Remington: The Science and Practice of Pharmacy, 23^{rd} edition, Oct. 30, 2020, Adeboye Adejare, ed. Accordingly, further provided is a pharmaceutical composition comprising the compound of formula I and a pharmaceutically acceptable carrier. In the compound of formula I, X can be Cl, F, I or Br. The compound of formula I can have the structure:

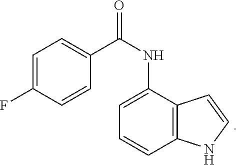 [SMILES: O=C(Nc1cccc2[nH]ccc12)c1ccc(F)cc1]

Still further provided is a pharmaceutical composition comprising the compound of formula II and a pharmaceutically acceptable carrier. In the compound of formula II, X can be Cl, F, I or Br.

In view of the above, provided is a method of inhibiting tau protein aggregation in a subject having, or at risk for, tau protein aggregation. The method comprises administering to the subject an above-described pharmaceutical composition in an amount effective to inhibit tau protein aggregation. The subject can have, or be at risk for, Alzheimer's disease.

Also in view of the above, provided is a method of inhibiting alpha-synuclein (α-syn) protein aggregation in a subject having, or at risk for, α-syn protein aggregation. The method comprises administering to the subject an above-described pharmaceutical composition in an amount effective to inhibit α-syn protein aggregation. The subject can have, or be at risk for, Parkinson's disease.

Any suitable route of administration can be used in the above methods. Examples include, but are not limited to, oral, parenteral, intravenous, intracranial, intracerebroventricular, and intracerebral. An effective amount can be determined by one of ordinary skill in the art using dosage range determining methods known in the art. Typically, a physician (or veterinarian for non-human subjects) will determine the actual dosage, which will be most suitable for an individual subject. The specific dose level and frequency of dosage for an individual may be varied and will depend upon a variety of factors including the activity of the specific compound employed, the metabolic stability and length of action of that compound, the age, body weight, general health, gender, diet, mode and time of administration, rate of excretion, other administered drugs, and the severity of the particular condition. The compound/compositions described herein can be administered with other biologically active compounds as appropriate.

The terms “substituted,” “substituent,” and “functional group” refer to a group that can be or is substituted onto a molecule or onto another group (e.g., on an aryl or an alkyl group). Examples of substituents include, but are not limited to, a halogen (e.g., F, Cl, Br, and I), OR, OC(O)N(R)₂, CN, NO, NO ONO₂, azido, CF₃, OCF₃, R, O (oxo), S (thiono), C(O), S(O), methylenedioxy, ethylenedioxy, N(R)₂, SR, SOR, SO₂R, SO₂N(R)₂, SO₃R, —(CH)₀₋₂P(O)(OR)₂, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)C(O)OR, (CH₂)₀₋₂N(R)N(R)₂, N(R)N(R)C(O)R, N(RN(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, or C(═NOR)R wherein each R can be, independently, hydrogen, alkyl, acyl, cycloalkyl, aryl, aralkyl, heterocyclyl, heteroaryl, or heteroarylalkyl, wherein any alkyl, acyl, cycloalkyl, aryl, aralkyl, heterocyclyl, heteroaryl, or heteroarylalkyl or two R groups bonded to a nitrogen atom or to adjacent nitrogen atoms can together with the nitrogen atom or atoms form a heterocyclyl, which can be mono- or independently multi-substituted.

The term “alkyl” as used herein refers to substituted or unsubstituted straight chain and branched mono- or divalent alkyl groups and cycloalkyl groups having from 1 to 40 carbon atoms (C₁-C₄₀), 1 to about 20 carbon atoms (C₁-C₂₀), 1 to 12 carbons (C₁-C₁₂), 1 to 8 carbon atoms (C₁-C₈), or, in some embodiments, from 1 to 6 carbon atoms (C₁-C₆). Examples of straight chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, t-butyl, neopentyl, isopentyl, and 2,2-dimethylpropyl groups. As used herein, the term “alkyl” encompasses n-alkyl, isoalkyl, and ante-isoalkyl groups as well as other branched chain forms of alkyl. Representative substituted alkyl groups can be substituted one or more times with any of the groups listed herein, for example, amino, hydroxy, cyano, carboxy, nitro, thio, alkoxy, and halogen groups.

The term “alkenyl” as used herein refers to substituted or unsubstituted straight chain and branched mono- or divalent alkenyl groups and cycloalkenyl groups having at least one double bond and having from 1 to 40 carbon atoms (C₁-C₄c), 1 to about 20 carbon atoms (C₁-C₂₀), 1 to 12 carbons (C₁-C₁₂), 1 to 8 carbon atoms (C₁-C₈), or, in some embodiments, from 1 to 6 carbon atoms (C₁-C₆). Examples of straight chain alkenyl groups include those with from 1 to 8 carbon atoms such as —CH═CH—, —CH═CHCH₃, and —CH₂CH═CHCH₂— groups, wherein the double bonds can have an E- or Z-configuration. And when there are multiple bonds, each double bond can, independently, have an E- or a Z-configuration. Examples of branched alkenyl groups include, but are not limited to, —CH═C(CH₃)— and CH₂C═CH(CH₃) groups. Representative substituted alkenyl groups can be substituted one or more times with any of the groups listed herein, for example, amino, hydroxy, cyano, carboxy, nitro, thio, alkoxy, and halogen groups.

The term “cycloalkyl” as used herein refers to substituted or unsubstituted cyclic alkyl groups such as, but not limited to, cyclopropyl, cyclobutyl, cyclopentyl, cyclohexyl, cycloheptyl, and cyclooctyl groups. In some embodiments, the cycloalkyl group can have 3 to about 8-12 ring members, whereas in other embodiments the number of ring carbon atoms range from 3 to 4, 5, 6, or 7. Cycloalkyl groups can have any number of carbon atoms, e.g., 3 to 8 carbon atoms (C₃-C₈), 3 to 6 carbon atoms (C₃-C₆), and 4 to 8 carbon atoms (C₄-C₈). Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like.

The term “cycloalkylalkyl” as used herein refers to substituted or unsubstituted alkyl groups as defined herein in which a hydrogen or carbon bond of an alkyl group as defined herein is replaced with a bond to a cycloalkyl group as defined herein. Representative cycloalkylalkyl groups include, but are not limited to, cyclopentylalkyl.

The term “alkylcycloalkyl” as used herein refers to substituted or unsubstituted cycloalkyl groups as defined herein in which a hydrogen of a cycloalkyl group as defined herein is replaced with a bond to an alkyl group as defined herein. Representative alkylcycloalkyl groups include, but are not limited to, alkylcyclopropyl.

The term “acyl” as used herein refers to a group containing a carbonyl moiety wherein the group is bonded via the carbonyl carbon atom. The carbonyl carbon atom is also bonded to another carbon atom, which can be part of a substituted or unsubstituted alkyl, aryl, aralkyl cycloalkyl, cycloalkylalkyl, heterocyclyl, heterocyclylalkyl, heteroaryl, heteroarylalkyl group or the like. In the special case wherein the carbonyl carbon atom is bonded to a hydrogen, the group is a “formyl” group, an acyl group as the term is defined herein. An acyl group can include 0 to about 12-40, 6-10, 1-5 or 2-5 additional carbon atoms bonded to the carbonyl group. An acryloyl group is an example of an acyl group. An acyl group can also include heteroatoms within the meaning here. A nicotinoyl group (pyridyl-3-carbonyl) is an example of an acyl group within the meaning herein. Other examples include acetyl, benzoyl, phenylacetyl, pyridylacetyl, cinnamoyl, and acryloyl groups and the like. When the group containing the carbon atom that is bonded to the carbonyl carbon atom contains a halogen, the group is termed a “haloacyl” group. An example is a trifluoroacetyl group.

The term “heterocyclylcarbonyl” is an example of an acyl group that is bonded to a substituted or unsubstituted heterocyclyl group, as the term “heterocyclyl” is defined herein. An example of a heterocyclylcarbonyl group is a prolyl group, wherein the prolyl group can be a D- or an L-prolyl group.

The term “aryl” as used herein refers to substituted or unsubstituted cyclic aromatic hydrocarbons that do not contain heteroatoms in the ring. Thus, aryl groups include, but are not limited to, phenyl, azulenyl, heptalenyl, biphenyl, indacenyl, fluorenyl, phenanthrenyl, triphenylenyl, pyrenyl, naphthacenyl, chrysenyl, biphenylenyl, anthracenyl, and naphthyl groups. In some embodiments, aryl groups contain about 6 to about 14 carbons (C₆-C₁₄) or from 6 to 10 carbon atoms (C₆-C₁₀) in the ring portions of the groups. Aryl groups can be unsubstituted or substituted, as defined herein. “Aryl” and the phrase “aryl group” includes fused ring species including those that include fused aromatic and non-aromatic groups. Accordingly, “aryl” and the phrase “aryl group” include groups of the formula:

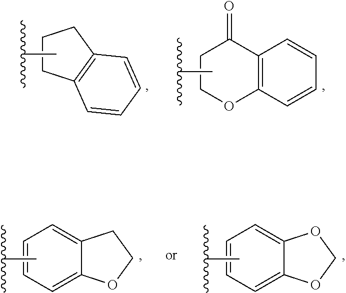

or each of which can be substituted or unsubstituted, such as hydroxy substituted.

Representative substituted aryl groups can be mono-substituted or substituted more than once, such as, but not limited to, 2-, 3-, 4-, 5-, or 6-substituted phenyl or 2-8 substituted naphthyl groups, which can be substituted with carbon or non-carbon groups such as those listed herein.

The terms “aralkyl” and “arylalkyl” refer to alkyl groups as defined herein in which a hydrogen or carbon bond of an alkyl group is replaced with a bond to an aryl group as defined herein. Representative aralkyl groups include benzyl and phenylethyl groups and fused (cycloalkylaryl)alkyl groups such as 4-ethyl-indanyl. Aralkenyl groups are alkenyl groups as defined herein in which a hydrogen or carbon bond of an alkyl group is replaced with a bond to an aryl group as defined herein.

The term “heterocyclyl” or “heterocyclo” refers to substituted or unsubstituted aromatic and non-aromatic ring compounds containing 3 or more ring members, of which one or more (e.g., 1, 2 or 3) is a heteroatom such as, but not limited to, N, O, and S. Thus, a heterocyclyl can be a cycloheteroalkyl or a heteroaryl or, if polycyclic, any combination thereof. In some embodiments, heterocyclyl groups include 3 to about 20 ring members, whereas other such groups have 3 to about 15 ring members. In some embodiments, heterocyclyl groups include heterocyclyl groups that include 3 to 8 carbon atoms (C₃-C₈), 3 to 6 carbon atoms (C₃-C₆), 3 to 5 carbon atoms (C₃-C₅) or 6 to 8 carbon atoms (C₆-C₈). A heterocyclyl group designated as a C₂-heterocyclyl can be a 5-ring with two carbon atoms and three heteroatoms, a 6-ring with two carbon atoms and the heteroatoms and so forth. Likewise, a C₄-heterocyclyl can be a 5-ring with one heteroatom, a 6-ring with two heteroatoms, and so forth. The number of carbon atoms plus the number of heteroatoms equals the total number of ring atoms. A heterocyclyl ring can also include one or more double bonds, such as in the group 3,6-dihydro-2H-pyran and 3,4-dihydro-2H-pyran, having the formula:

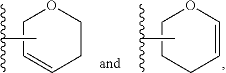

respectively, each of which can be substituted.

A heteroaryl ring is an embodiment of a heterocyclyl group. The phrase “heterocyclyl group” includes fused ring species including those that include fused aromatic and non-aromatic groups. Representative heterocyclyl groups include, but are not limited to tetrahydro-2H-thiopyran-1,1-dioxide, having the formula:

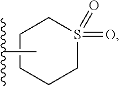

which can be substituted, 4a,5,6,7-tetrahydro-4H-pyrrolo[1,2-d][1,3,4]oxadiazinyi, having the formula:

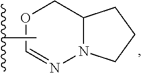

which can be substituted, pyrrolidinyl, pyrrolidinone (e.g., pyrrolidin-2-one), azetidinyl, piperidynyl, piperazinyl, morpholinyl, chromanyl, indolinonyl, isoindolinonyl, furanyl, pyrrolidinyl, pyridinyl, pyrazinyl, pyrimidinyl, triazinyl, thiophenyl, tetrahydrofuranyl, pyrrolyl, oxazolyl, oxadiazolyl, imidazolyl, imidazo[1,2-a]pyridinyl, having the formula:

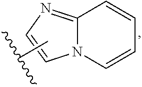

which can be substituted, triazyolyl, tetrazolyl, benzoxazolinyl, thiazolyl, benzthiazolinyl, and benzimidazolinyl groups. Examples of indolinonyl groups include groups having the general formula:

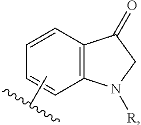

wherein R is as defined herein.

Examples of isoindolinonyl groups include groups having the general formula:

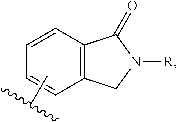

wherein R is as defined herein.

Examples of benzoxazolinyl groups include groups having the general formula:

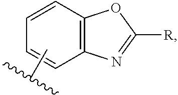

wherein R is as defined herein.

Examples of benzthiazolinyl groups include groups having the general formula:

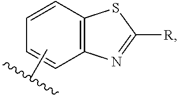

wherein R is as defined herein.

In some embodiments, the group R in benzoxazolinyl and benzthiazolinyl groups is an N(R)₂ group. In some embodiments, each R is hydrogen or alkyl, wherein the alkyl group is substituted or unsubstituted. In some embodiments, the alkyl group is substituted with a heterocyclyl group (e.g., with a pyrrolidinyl group).

The term “heterocyclylalkyl” refers to alkyl groups as defined herein in which a hydrogen or carbon bond of an alkyl group as defined herein is replaced with a bond to a heterocyclyl group as defined herein. Representative heterocyclylalkyl groups include, but are not limited to, furan-2-yl methyl, furan-3-yl methyl, pyridine-3-yl methyl, tetrahydrofuran-2-yl methyl, and indol-2-yl propyl.

The term “heterocyclylalkoxy” refers to alkyl groups as defined herein in which a hydrogen or carbon bond of an alkyl group as defined herein is replaced with a bond to a heterocyclyl group as defined herein and the alkyl group is attached to an oxygen. Representative heterocyclylalkoxy groups include, but are not limited to, —O—(CH₂)_{q}heterocyclyl, wherein q is an integer from 1 to 5. In some embodiments, heterocyclylalkoxy groups include —O—(CH₂)_{q}morpholinyl such as —O—CH₂CH₂-morpholine.

The term “heteroarylalkyl” refers to alkyl groups as defined herein in which a hydrogen or carbon bond of an alkyl group is replaced with a bond to a heteroaryl group as defined herein.

The term “alkoxy” refers to an oxygen atom connected to an alkyl group, including a cycloalkyl group, as are defined herein. Examples of linear alkoxy groups include, but are not limited to, methoxy, ethoxy, propoxy, butoxy, pentyloxy, hexyloxy, and the like. Examples of branched alkoxy include, but are not limited to, isopropoxy, sec-butoxy, tert-butoxy, isopentyloxy, isohexyloxy, and the like. Examples of cyclic alkoxy include, but are not limited to, cyclopropyloxy, cyclobutyloxy, cyclopentyloxy, cyclohexyloxy, and the like. An alkoxy group can include one to about 12-20 or about 12-40 carbon atoms bonded to the oxygen atom, can further include double or triple bonds, and can also include heteroatoms. For example, an allyloxy group is an alkoxy group within the meaning herein. A methoxyethoxy group is also an alkoxy group within the meaning herein, as is a methylenedioxy group in a context where two adjacent atoms of a structure are substituted therewith.

The terms “amine,” “amine group,” “amino,” and “amino group” refer to a substituent of the form —NH₂—NHR, —NR₂) or —NR₃⁺, wherein each R is defined herein, and protonated forms of each, except for —NR₃⁺, which cannot be protonated. Accordingly, any compound substituted with an amino group can be viewed as an amine. An “amino group” within the meaning herein can be a primary, secondary, tertiary, or quaternary amino group.

An “alkylamino” group includes a monoalkylamino, dialkylamino, and trialkylamino group. An example of a “alkylamino” is —NH-alkyl and —N(alkyl)₂.

An example of a “cycloalkylamino” group is —NH-cycloalkyl and —N(cycloalkyl)₂.

An example of a “cycloalkyl heterocycloamino” group is —NH-(heterocyclo cycloalkyl), wherein the heterocyclo group is attached to the nitrogen and the cycloalkyl group is attached to the heterocyclo group.

An example of a “heterocyclo cycloamino” group is —NH-(cycloalkyl heterocycle), wherein the cycloalkyl group is attached to the nitrogen and the heterocyclo group is attached to the cycloalkyl group.

The term “amido” refers to a group of the formula —C(O)NR₂, wherein R is defined herein.

The terms “halo,” “halogen,” and “halide” group, by themselves or as part of another substituent, mean, unless otherwise stated, a fluorine, chlorine, bromine, or iodine atom.

The term “haloalkyl” group includes mono-halo alkyl groups, poly-halo alkyl groups, wherein all halo atoms can be the same or different, and per-halo alkyl groups, wherein all hydrogen atoms are replaced by halogen atoms, such as fluoro. Examples of haloalkyl include trifluoromethyl, 1,1-dichloroethyl, 1,2-dichloroethyl, 1,3-dibromno-3,3-difluoropropyl, perfluorobutyl, —CF(CH₃) and the like.

The terms “treat,” “treating,” “treated,” or “treatment” (with respect to a disease or condition) is an approach for obtaining beneficial or desired results including and preferably clinical results and includes, but is not limited to, one or more of the following: improving a condition associated with a disease, curing a disease, lessening severity of a disease, delaying progression of a disease, alleviating one or more symptoms associated with a disease, increasing the quality of life of one suffering from a disease, prolonging survival and/or prophylactic or preventative treatment.

An “effective amount” refers to any amount that is sufficient to achieve a desired biological effect. Combined with the teachings provided herein, by choosing among the various active conjugates or compounds and weighing factors such as potency, relative bioavailability, patient body weight, severity of adverse side-effects and mode of administration, an effective prophylactic or therapeutic treatment regimen can be planned which does not cause substantial unwanted toxicity and yet is effective to treat the particular subject. The effective amount for any particular application can vary depending on such factors as the disease or condition being treated, the particular compound being administered, the size of the subject, or the severity of the disease or condition, One of ordinary skill in the art can empirically determine the effective amount of a particular compound and/or other therapeutic agent without necessitating undue experimentation. A maximum dose can be used, that is, the highest safe dose according to some medical judgment. Multiple doses per day can be used to achieve appropriate systemic levels of compounds. Appropriate systemic levels can be determined by, for example, measurement of the patient's peak or sustained plasma level of the drug. “Dose” and “dosage” are used interchangeably herein.

Generally, daily oral doses of a compound are, for human subjects, from about 0.01 milligrams/kg per day to 1,000 milligrams/kg per day. Oral doses in the range of 0.5 to 50 milligrams/kg, in one or more administrations per day, can yield therapeutic results. Dosage can be adjusted appropriately to achieve desired drug levels, local or systemic, depending upon the mode of administration. For example, intravenous administration can vary from one order to several orders of magnitude lower dose per day. If the response in a subject is insufficient at such doses, even higher doses (or effective higher doses by a different, more localized delivery route) can be employed to the extent that patient tolerance permits. Multiple doses per day are contemplated to achieve appropriate systemic levels of the compound.

A “therapeutically effective amount” (or “effective amount”) of a compound with respect to use in treatment, refers to an amount of the compound in a preparation which, when administered as part of a desired dosage regimen (to a mammal, such as a human) alleviates a symptom, ameliorates a condition, or slows the onset of disease conditions according to clinically acceptable standards for the disorder or condition to be treated or the cosmetic purpose, e.g., at a reasonable benefit/risk ratio applicable to any medical treatment.

For any compound a therapeutically effective amount can be initially determined from animal models. A therapeutically effective dose can also be determined from human data for compounds which have been tested in humans and for compounds which are known to exhibit similar pharmacological activities, such as other related active agents. Higher doses may be required for parenteral administration. The applied dose can be adjusted based on the relative bioavailability and potency of the administered compound. Adjusting the dose to achieve maximal efficacy based on the methods described above and other methods as are well-known in the art is well within the capabilities of the ordinarily skilled artisan.

The formulations can be administered in pharmaceutically acceptable solutions, which can routinely contain pharmaceutically acceptable concentrations of salt, buffering agents, preservatives, compatible carriers, adjuvants, and optionally other therapeutic ingredients. For use in therapy, an effective amount of the compound can be administered to a subject by any mode that delivers the compound to the desired surface. Administering a pharmaceutical composition can be accomplished by any means known to the skilled artisan. Routes of administration include, but are not limited to, intravenous, intramuscular, intraperitoneal, intravesical (urinary bladder), oral, subcutaneous, direct injection (for example, into a tumor or abscess), mucosal (e.g., topical to eye), inhalation, and topical.

For intravenous and other parenteral routes of administration, a compound can be formulated as a lyophilized preparation, as a lyophilized preparation of liposome-intercalated or -encapsulated active compound, as a lipid complex in aqueous suspension, or as a salt complex. Lyophilized formulations are generally reconstituted in suitable aqueous solution, e.g., in sterile water or saline, shortly prior to administration.

For oral administration, the compounds can be formulated readily by combining the active compound(s) with pharmaceutically acceptable carriers well-known in the art. Such carriers enable the compounds to be formulated as tablets, pills, dragees, capsules, liquids, gels, syrups, slurries, suspensions and the like, for oral ingestion by a subject to be treated. A pharmaceutical preparation for oral use can be obtained as a solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, and sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinyl pyrrolidone (PVP). If desired, disintegrating agents can be added, such as the cross-linked PVP, agar, or alginic acid or a salt thereof such as sodium alginate. Optionally the oral formulations can also be formulated in saline or buffers, e.g., EDTA for neutralizing internal acid conditions, or can be administered without any carriers.

Also contemplated are oral dosage forms of the compounds. The compounds can be chemically modified so that oral delivery of the derivative is efficacious. Generally, the chemical modification contemplated is the attachment of at least one moiety to the compound itself, where said moiety permits (a) inhibition of acid hydrolysis; and (b) uptake into the blood stream from the stomach or intestine. Also desired is the increase in overall stability of the compounds and increase in circulation time in the body. Examples of such moieties include polyethylene glycol, copolymers of ethylene glycol and propylene glycol, carboxymethyl cellulose, dextran, polyvinyl alcohol, PVP and polyproline. Abuchowski and Davis, “Soluble Polymer-Enzyme Adducts,” In: Enzymes as Drugs, Hocenberg and Roberts, eds., Wiley-Interscience, New York, N.Y., pp. 367-383 (1981); Newmark et al., J Appl Biochem 4:185-189 (1982). Other polymers that could be used are poly-1,3-dioxolane and poly-1,3,6-tioxocane. For pharmaceutical usage, as indicated above, polyethylene glycol moieties are suitable.

The location of release of a compound hereof can be the stomach, the small intestine (e.g., the duodenum, the jejunum, or the ileum), or the large intestine. One skilled in the art has available formulations, which will not dissolve in the stomach, yet will release the material in the duodenum or elsewhere in the intestine. The release can avoid the deleterious effects of the stomach environment, either by protection of the compound or by release of the compound beyond the stomach environment, such as in the intestine.

To ensure full gastric resistance a coating impermeable to at least pH 5.0 is essential. Examples of the more common inert ingredients that are used as enteric coatings are cellulose acetate trimellitate (CAT), hydroxypropylmethyicellulose phthalate (HPMCP), HPMCP 50, HPMCP 55, polyvinyl acetate phthalate (PVAP), Eudragit L30D, Aquateric, cellulose acetate phthalate (CAP), Eudragit L, Eudragit S, and shellac. These coatings can be used as mixed films.

A coating or mixture of coatings can also be used on tablets, which are not intended for protection against the stomach. This can include sugar coatings, or coatings which make the tablet easier to swallow. Capsules can consist of a hard shell (such as gelatin) for delivery of dry therapeutic (e.g., powder); for liquid forms, a soft gelatin shell can be used. The shell material of cachets could be thick starch or other edible paper. For pills, lozenges, molded tablets or tablet triturates, moist massing techniques can be used.

The compound can be included in the formulation as fine multi-particulates in the form of granules or pellets of particle size about 1 mm. The formulation of the material for capsule administration could also be as a powder, lightly compressed plugs or even as tablets. Therapeutic agent could be prepared by compression.

Colorants and flavoring agents may all be included. For example, the compound can be formulated (such as by liposome or microsphere encapsulation) and then further contained within an edible product, such as a refrigerated beverage containing colorants and flavoring agents.

One may dilute or increase the volume of the compound with an inert material. These diluents can include carbohydrates, especially mannitol, a-lactose, anhydrous lactose, cellulose, sucrose, modified dextrans and starch. Certain inorganic salts also can be used as fillers including calcium triphosphate, magnesium carbonate and sodium chloride. Some commercially available diluents are Fast-Flo, Emdex, STA-Rx 1500, Emcompress and Avicell.

Disintegrants can be included in the formulation of therapeutic agent into a solid dosage form. Materials used as disintegrates include, but are not limited to, starch, including the commercial disintegrant based on starch, Explotab. Sodium starch glycolate, Amberlite, sodium carboxymethylcellulose, ultramylopectin, sodium alginate, gelatin, orange peel, acid carboxymethyl cellulose, natural sponge and bentonite may all be used. Another form of the disintegrant is the insoluble cationic exchange resin. Powdered gums can be used as disintegrants and as binders, and these can include powdered gums such as agar, Karaya or tragacanth. Alginic acid and its sodium salt are also useful as disintegrants.

Binders can be used to hold the compound together to form a hard tablet and can include materials from natural products such as acacia, tragacanth, starch and gelatin. Others include methyl cellulose (MC), ethyl cellulose (EC) and carboxymethyl cellulose (CMC). PVP and hydroxypropylmethyl cellulose (HPMC) can both be used in alcoholic solutions to granulate therapeutic agent.

An anti-frictional agent can be included in the formulation of therapeutic to prevent sticking during the formulation process. Lubricants can be used as a layer between therapeutic agent and the die wall, and these can include, but are not limited to, stearic acid, including its magnesium and calcium salts, polytetrafluoroethylene (PTFE), liquid paraffin, vegetable oils and waxes. Soluble lubricants can also be used, such as sodium lauryl sulfate, magnesium lauryl sulfate, polyethylene glycol of various molecular weights, Carbowax 4000 and 6000.

Glidants, which can improve the flow properties of the drug during formulation and aid rearrangement during compression, can be added. The glidants can include starch, talc, pyrogenic silica and hydrated silicoaluminate.

To aid dissolution of therapeutic agent into the aqueous environment a surfactant can be added as a wetting agent. Surfactants can include anionic detergents, such as sodium lauryl sulfate, dioctyl sodium sulfosuccinate and dioctyl sodium sulfonate. Cationic detergents, which can be used, include benzalkonium chloride and benzethonium chloride. Potential non-ionic detergents that can be included in the formulation as surfactants include lauromacrogol 400, polyoxyl 40 stearate, polyoxyethylene hydrogenated castor oil 10, 50 and 60, glycerol monostearate, polysorbate 40, 60, 65 and 80, sucrose fatty acid ester, menthyl cellulose and carboxymethyl cellulose. These surfactants could be present in the formulation of the compound or derivative thereof either alone or as a mixture in different ratios.

Pharmaceutical preparations, which can be used orally, include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds can be dissolved or suspended in suitable liquids, such as fatty oils, liquid paraffin, or liquid polyethylene glycols. In addition, stabilizers can be added. Microspheres formulated for oral administration can also be used. Such microspheres have been well-defined in the art. All formulations for oral administration should be in dosages suitable for such administration.

For buccal administration, the compositions can take the form of tablets or lozenges formulated in conventional manner.

For topical administration, the compound can be formulated as solutions, gels, ointments, creams, suspensions, etc. as are well-known in the art. Systemic formulations include those designed for administration by injection, e.g., subcutaneous, intravenous, intramuscular, intrathecal or intraperitoneal injection, as well as those designed for transdermal, transmucosal oral or pulmonary administration.

For administration by inhalation, compounds can be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of e.g., gelatin for use in an inhaler or insufflator, can be formulated containing a powder mix of the compound and a suitable powder base, such as lactose or starch.

Also contemplated is pulmonary delivery of the compounds (or salts thereof). The compound is delivered to the lungs of a mammal while inhaling and traverses across the lung epithelial lining to the blood stream. Other reports of inhaled molecules include Adjei et al., Pharm Res 7:565-569 (1990); Adjei et al., Int J Pharmaceutics 63:135-144 (1990) (leuprolide acetate); Braquet et al., J Cardiovasc Pharmacol 13(suppl. 5):143-146 (1989) (endothelin-!); Hubbard et al., Annal Int Med 3:206-212 (1989) (al-antitrypsin); Smith et al., 1989, J Clin Invest 84:1145-1146 (a-1-proteinase); Oswein et al., 1990, “Aerosolization of Proteins,” Proceedings of Symposium on Respiratory Drug Delivery II, Keystone, Colorado, March, (recombinant hepatocyte growth hormone); Debs et al., 1988, J Immunol 140:3482-3488 (interferon-gamma and tumor necrosis factor alpha) and Platz et al., U.S. Pat. No. 5,284,656 (granulocyte colony stimulating factor; incorporated herein by reference). A method and composition for pulmonary delivery of drugs for systemic effect is described in U.S. Pat. No. 5,451,569 (specifically incorporated herein by reference for its disclosure regarding same), issued Sep. 19, 1995, to Wong et al.

Contemplated for use are a wide range of mechanical devices designed for pulmonary delivery of therapeutic products including, but not limited to, nebulizers, metered dose inhalers, and powder inhalers, all of which are familiar to those skilled in the art.

Nasal delivery of a pharmaceutical composition is also contemplated. Nasal delivery allows the passage of a pharmaceutical composition to the blood stream directly after administering therapeutic product to the nose, without the necessity for deposition of the product in the lung. Formulations for nasal delivery include those with dextran or cyclodextran.

The compounds, when it is desirable to deliver them systemically, can be formulated for parenteral administration by injection, e.g., by bolus injection or continuous infusion. Formulations for injection can be presented in unit dosage form, e.g., in ampoules or in multi-dose containers, with an added preservative. The compositions can take such forms as suspensions, solutions or emulsions in oily or aqueous vehicles, and can contain formulatory agents such as suspending, stabilizing and/or dispersing agents.

Pharmaceutical formulations for parenteral administration include aqueous solutions of the active compounds in water-soluble form, Additionally, suspensions of the active compounds can be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions can contain substances which increase the viscosity of the suspension, such as sodium carboxymethylcellulose, sorbitol, or dextran. Optionally, the suspension can also contain suitable stabilizers or agents which increase the solubility of the compounds to allow for the preparation of highly concentrated solutions.

Alternatively, the active compounds can be in powder form for constitution with a suitable vehicle, e.g., sterile pyrogen-free water, before use.

The compounds can also be formulated in rectal or vaginal compositions such as suppositories or retention enemas, e.g., containing conventional suppository bases such as cocoa butter or other glycerides.

In addition to the formulations described above, a compound can also be formulated as a depot preparation. Such long-acting formulations can be formulated with suitable polymeric or hydrophobic materials (for example as an emulsion in an acceptable oil) or ion exchange resins, or as sparingly soluble derivatives, for example, as a sparingly soluble salt.

The pharmaceutical compositions also can comprise suitable solid or gel phase carriers or excipients. Examples of such carriers or excipients include, but are not limited to, calcium carbonate, calcium phosphate, various sugars, starches, cellulose derivatives, gelatin, and polymers such as polyethylene glycols.

Suitable liquid or solid pharmaceutical preparation forms are, for example, aqueous or saline solutions for inhalation, microencapsulated, encochleated, coated onto microscopic gold particles, contained in liposomes, nebulized, aerosolized, pelleted for implantation into the skin, or dried onto a sharp object to be scratched into the skin. The pharmaceutical compositions also include granules, powders, tablets, coated tablets, (micro)capsules, suppositories, syrups, emulsions, suspensions, creams, drops or preparations with protracted release of active compounds, in whose preparation excipients and additives and/or auxiliaries such as disintegrants, binders, coating agents, swelling agents, lubricants, flavorings, sweeteners or solubilizers are customarily used as described above. The pharmaceutical compositions are suitable for use in a variety of drug delivery systems. For a brief review of methods for drug delivery, see Langer R, Science 249:1527-1533 (1990).

The compound and optionally one or more other therapeutic agents can be administered per se (neat) or in the form of a pharmaceutically acceptable salt. When used in medicine the salts should be pharmaceutically acceptable, but non-pharmaceutically acceptable salts may conveniently be used to prepare pharmaceutically acceptable salts thereof, Such salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, such salts can be prepared as alkaline metal or alkaline earth salts, such as sodium, potassium or calcium salts of the carboxylic acid group.

Suitable buffering agents include acetic acid and a salt (1-2% w/v); citric acid and a salt (1-3% w/v); boric acid and a salt (0.5-2.5% w/v); and phosphoric acid and a salt (0.8-2% w/v). Suitable preservatives include benzalkonium chloride (0.003-0.03% w/v); chlorobutanol (0.3-0.9% w/v); parabens (0.01-0.25% w/v); and thimerosal (0.004-0.02% w/v).

Pharmaceutical compositions contain an effective amount of a compound as described herein and optionally one or more other therapeutic agents included in a pharmaceutically acceptable carrier. The term “pharmaceutically acceptable carrier” means one or more compatible solid or liquid fillers, diluents or encapsulating substances, which are suitable for administration to a human or other vertebrate animal. The term “carrier” denotes an organic or inorganic ingredient, natural or synthetic, with which the active ingredient is combined to facilitate the application. The components of the pharmaceutical compositions also can be commingled with the compounds, and with each other, in a manner such that there is no interaction, which would substantially impair the desired pharmaceutical efficiency.

Therapeutic agent(s), including specifically, but not limited to, a compound, can be provided in particles. “Particles” means nanoparticles or microparticles (or in some instances larger particles) that can consist in whole or in part of the compound or the other therapeutic agent(s). The particles can contain therapeutic agent(s) in a core surrounded by a coating, including, but not limited to, an enteric coating. Therapeutic agent(s) also can be dispersed throughout the particles. Therapeutic agent(s) also can be adsorbed into the particles. The particles can be of any order release kinetics, including zero-order release, first-order release, second-order release, delayed release, sustained release, immediate release, and any combination thereof, etc. The particle can include, in addition to therapeutic agent(s), any of those materials routinely used in the art of pharmacy and medicine, including, but not limited to, erodible, nonerodible, biodegradable, or nonbiodegradable material or combinations thereof. The particles can be microcapsules which contain the compound in a solution or in a semi-solid state. The particles can be of virtually any shape.

Both non-biodegradable and biodegradable polymeric materials can be used in the manufacture of particles for delivering therapeutic agent(s). Such polymers can be natural or synthetic polymers. The polymer is selected based on the period of time over which release is desired. Bioadhesive polymers of particular interest include bioerodible hydrogels described in Sawhney et al., Macromolecules 26:5823-2787 (1993), the teachings of which are specifically incorporated by reference herein. These include polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate).

Therapeutic agent(s) can be contained in controlled-release systems. The term “controlled release” refers to any drug-containing formulation in which the manner and profile of drug release from the formulation are controlled. This refers to immediate as well as non-immediate release formulations, with non-immediate release formulations including, but not limited to, sustained release and delayed release formulations. The term “sustained release” (also referred to as “extended release”) refers to a drug formulation that provides for gradual release of a drug over an extended period of time, and that can result in substantially constant blood levels of a drug over an extended time period. The term “delayed release” refers to a drug formulation in which there is a time delay between administration of the formulation and the release of the drug therefrom. “Delayed release” may or may not involve gradual release of drug over an extended period of time, and thus may or may not be “sustained release.”

Use of a long-term, sustained-release implant can be particularly suitable for treatment of chronic conditions. “Long-term” release means that the implant is constructed and arranged to deliver therapeutic levels of the active ingredient for at least 7 days, and up to 30-60 days. Long-term sustained-release implants are well-known to those of ordinary skill in the art and include some of the release systems described above.

The term “salts” and “pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic groups such as amines; and alkali or organic salts of acidic groups such as carboxylic acids. Pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, and nitric; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, and isethionic, and the like.

Pharmaceutically acceptable salts can be synthesized from the parent compound, which contains a basic or acidic moiety, by conventional chemical methods. In some instances, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 18th ed., Mack Publishing Company, Easton, Pa., 1990, the disclosure of which is hereby incorporated by reference.

The term “solvate” means a compound, or a salt thereof, that further includes a stoichiometric or non-stoichiometric amount of solvent bound by non-covalent intermolecular forces. Where the solvent is water, the solvate is a hydrate.

The term “prodrug” means a derivative of a compound that can hydrolyze, oxidize, or otherwise react under biological conditions (in vitro or in vivo) to provide an active compound, particularly a compound of the invention, Examples of prodrugs include, but are not limited to, derivatives and metabolites of a compound of the invention that include biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. Specific prodrugs of compounds with carboxyl functional groups are the lower alkyl esters of the carboxylic acid. The carboxylate esters are conveniently formed by esterifying any of the carboxylic acid moieties present on the molecule. Prodrugs can typically be prepared using well-known methods, such as those described by Burger's Medicinal Chemistry and Drug Discovery 6th ed. (Donald J. Abraham ed., 2001, Wiley) and Design and Application of Prodrugs (I. Bundgaard ed., 1985, Harwood Academic Publishers GmbH).

Further, in each of the foregoing and following embodiments, it is to be understood that the formulae include and represent not only all pharmaceutically acceptable salts of the compounds, but also include any and all hydrates and/or solvates of the compound formulae or salts thereof. It is to be appreciated that certain functional groups, such as the hydroxy, amino, and like groups form complexes and/or coordination compounds with water and/or various solvents, in the various physical forms of the compounds. Accordingly, the above formulae are to be understood to include and represent those various hydrates and/or solvates. In each of the foregoing and following embodiments, it is also to be understood that the formulae include and represent each possible isomer, such as stereoisomers and geometric isomers, both individually and in any and all possible mixtures. In each of the foregoing and following embodiments, it is also to be understood that the formulae include and represent any and all crystalline forms, partially crystalline forms, and non-crystalline and/or amorphous forms of the compounds.

The term “pharmaceutically acceptable carrier” is art-recognized and refers to a pharmaceutically acceptable material, composition or vehicle, such as a liquid or solid filler, diluent, excipient, solvent or encapsulating material, involved in carrying or transporting any subject composition or component thereof. Each carrier must be “acceptable” in the sense of being compatible with the subject composition and its components and not injurious to the patient. Some examples of materials, which may serve as pharmaceutically acceptable carriers, include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The term “administering” includes all means of introducing the compounds and compositions described herein to the patient, including, but are not limited to, oral (po), intravenous (iv), intramuscular (im), subcutaneous (sc), transdermal, inhalation, buccal, ocular, sublingual, vaginal, rectal, and the like. The compounds and compositions may be administered in unit dosage forms and/or formulations containing conventional nontoxic pharmaceutically acceptable carriers, adjuvants, and vehicles.

Illustrative formats for oral administration include tablets, capsules, elixirs, syrups, and the like. Illustrative routes for parenteral administration include intravenous, intraarterial, intraperitoneal, epidural, intraurethral, intrasternal, intramuscular and subcutaneous, as well as any other art recognized route of parenteral administration.

Illustrative means of parenteral administration include needle (including microneedle) injectors, needle-free injectors and infusion techniques, as well as any other means of parenteral administration recognized in the art. Parenteral formulations are typically aqueous solutions, which may contain excipients such as salts, carbohydrates and buffering agents (preferably at a pH in the range from about 3 to about 9), but, for some applications, they may be more suitably formulated as a sterile non-aqueous solution or as a dried form to be used in conjunction with a suitable vehicle such as sterile, pyrogen-free water. The preparation of parenteral formulations under sterile conditions, for example, by lyophilization, may readily be accomplished using standard pharmaceutical techniques well-known to those skilled in the art. Parenteral administration of a compound is illustratively performed in the form of saline solutions or with the compound incorporated into liposomes. In cases where the compound, itself, is not sufficiently soluble to be dissolved, a solubilizer such as ethanol can be applied.

The dosage of each compound of the claimed combinations depends on several factors, including: the administration method, the condition to be treated, the severity of the condition, whether the condition is to be treated or prevented, and the age, weight, and health of the person to be treated. Additionally, pharmacogenomic (the effect of genotype on the pharmacokinetic, pharmacodynamic or efficacy profile of a therapeutic) information about a particular patient may affect the dosage regimen used.

In the methods the individual components of a co-administration, or combination, can be administered by any suitable means, contemporaneously, simultaneously, sequentially in either order, separately or in a single pharmaceutical formulation. Where the co-administered compounds or compositions are administered in separate dosage forms, the number of dosages administered per day for each compound may be the same or different. The compounds or compositions may be administered via the same or different routes of administration. The compounds or compositions may be administered according to simultaneous or alternating regimens, at the same or different times during the course of the therapy, concurrently in divided or single forms.

The term “therapeutically effective amount” refers to that amount of active compound or pharmaceutical agent that elicits the biological or medicinal response in a tissue system, animal or human that is being sought by a researcher, veterinarian, medical doctor or other clinician, which includes alleviation of the symptoms of the disease or disorder being treated. In one aspect, the therapeutically effective amount is that which may treat or alleviate the disease or symptoms of the disease at a reasonable benefit/risk ratio applicable to any medical treatment. However, it is to be understood that the total daily usage of the compounds and compositions described herein may be decided by the attending physician within the scope of sound medical judgment. The specific therapeutically effective dose level for any particular patient will depend upon a variety of factors, including the disorder being treated and the severity of the disorder; activity of the specific compound employed; the specific composition employed; the age, body weight, general health, gender and diet of the patient; the time of administration, route of administration, and rate of excretion of the specific compound employed; the duration of the treatment; drugs used in combination or coincidentally with the specific compound employed; and like factors well-known to the researcher, veterinarian, medical doctor or other clinician of ordinary skill.

Depending upon the route of administration, a wide range of permissible dosages are contemplated, including doses falling in the range from about 1 μg/kg to about 1 g/kg. The dosages may be single or divided and may administered according to a wide variety of protocols, including q.d. (once a day), b.i.d. (twice a day), t.i.d. (three times a day), or even every other day, once a week, once a month, once a quarter, and the like. In each of these cases the described therapeutically effective amounts correspond to the instance of administration, or alternatively to the total daily, weekly, month, or quarterly dose, as determined by the dosing protocol.

An effective amount of any one or a mixture of the compounds can be determined by the attending diagnostician or physician by the use of known techniques and/or by observing results obtained under analogous circumstances. In determining the effective amount or dose, a number of factors are considered by the attending diagnostician or physician, including, but not limited to, the species of mammal, including human, its size, age, and general health, the specific disease or disorder involved, the degree of or involvement or the severity of the disease or disorder, the response of the individual patient, the particular compound administered, the mode of administration, the bioavailability characteristics of the preparation administered, the dose regimen selected, the use of concomitant medication, and other relevant circumstances.

The term “patient” includes human and non-human animals such as companion animals (dogs and cats and the like) and livestock animals. Livestock animals are animals raised for food production. The patient to be treated is preferably a mammal, in particular a human.

The disclosure relates to, among other things, the following enumerated Embodiments, which listing does not represent an order of importance:

Embodiment 1. A compound of the formula:

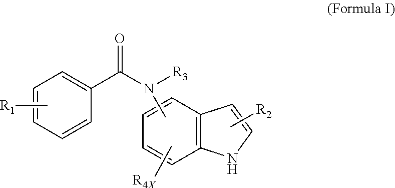

or a pharmaceutically acceptable salt thereof,
wherein R₁ is H, RₐO—, N≡C—, NO₂, NH₂, X₃C— or X, Rₐ is C₁-C₆ alkyl, and X is a halo;
R₂ is C₁-C₆ alkyl or X, wherein X is a halo,
R₃ is H or C₁-C₆ alkyl or X, wherein X is a halo; and
R₄ₓ, in which x is 1, 2 or 3, each R₄ is independently H, C₁-C₆ alkyl, or X, wherein X is a halo.

Embodiment 2. The compound of Embodiment 1, or a pharmaceutically acceptable salt thereof, wherein X is Cl, F, I or Br.

Embodiment 3. The compound of Embodiment 1, having the structure:

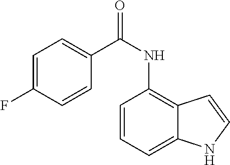 [SMILES: O=C(Nc1cccc2[nH]ccc12)c1ccc(F)cc1]

or a pharmaceutically acceptable salt thereof.

Embodiment 4. A compound of the formula:

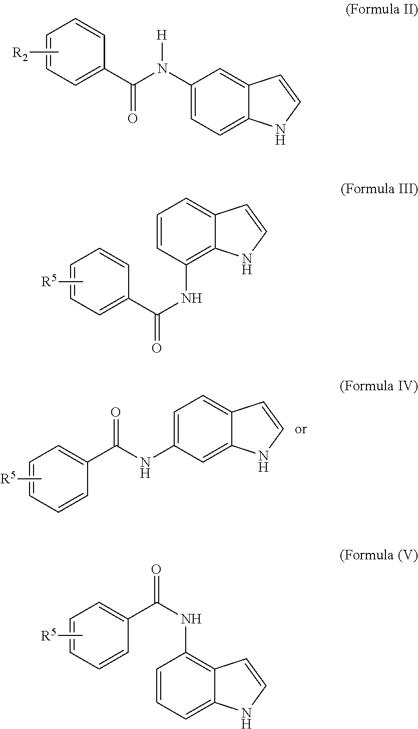

or a pharmaceutically acceptable salt thereof
wherein R₂ is H, R^{b}O—, N≡C—, NO₂, NH₂, X₃C— or X,
R_{U} is a C₁-C₆ alkyl, and
X is a halo.

Embodiment 5. The compound of Embodiment 4, or a pharmaceutically acceptable salt thereof, wherein X is Cl, F, I or Br.

Embodiment 6. A compound of the formula:

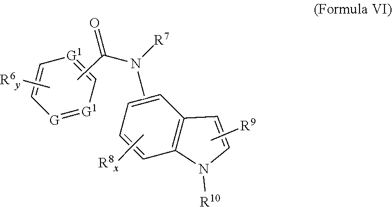

or a pharmaceutically acceptable salt thereof,
wherein:
G is N, CR⁶ or absent:
each C is independently N, S, CR;
each R⁶ is independently H, alkyl, alkoxy, aryl, aryloxy, alkoxy, N≡C—, NO₂, amino, X₃C— or halo or two R⁶ form a cycloalkyl group, an arylcycloalkyl group, an aryl group or a heterocyclyl group;
y is 1, 2, 3, or 4;
R⁷ is H or C₁-C₆ alkyl;
each R⁸ is C₁-C₆ alkyl or X, wherein X is a halo;
x is 1, 2 or 3; and
R⁹ is H, C₁-C₆ alkyl or X, wherein X is a halo.

Embodiment 7. The compound of Embodiment 6, or a pharmaceutically acceptable salt thereof, wherein the compound is a compound of the formula:

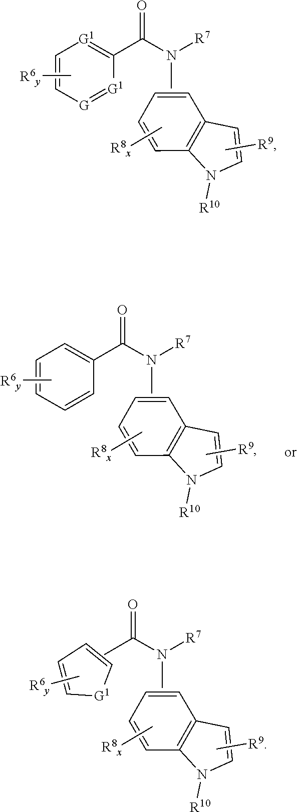

Embodiment 8. The compound of Embodiment 6, or a pharmaceutically acceptable salt thereof, wherein the compound is a compound of the formula:

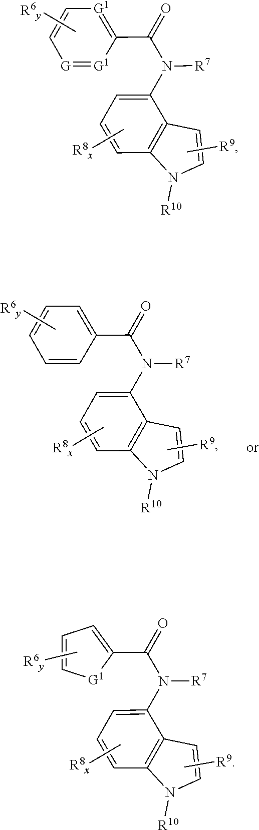

Embodiment 9. The compound of any one of Embodiments 6-8, or a pharmaceutically acceptable salt thereof, wherein each G¹ is CR⁶, each G is CR⁶, or both G¹ are G and CR⁶.

Embodiment 10. The compound of any one of Embodiments 6-8, or a pharmaceutically acceptable salt thereof, wherein the group of the formula:

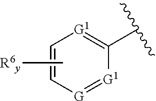

is a group of the formula:

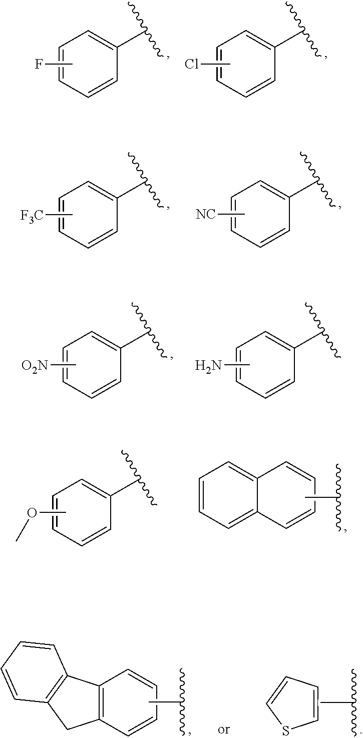

Embodiment 11. The coumpund of Embodiment 9, or a pharmaceutically acceptable salt thereof, wherein the group of formua:

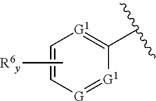

is a group of the formula:

Embodiment 12. The compound of any one of Embodiments 6-8, or a

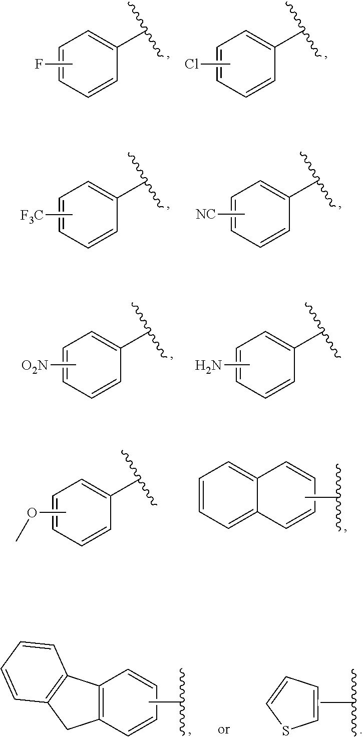

pharmaceutically acceptable salt thereof, wherein R⁷ and/or R¹⁰ is C₁-C₃ alkyl.

Embodiment 13. The compound of Embodiment 9, or a pharmaceutically acceptable salt thereof, wherein R⁷ and/or R¹⁰ is C₁-C₃ alkyl.

Embodiment 14. The compound of Embodiment 10, or a pharmaceutically acceptable salt thereof, wherein R⁷ and/or R¹⁰ is C₁-C₃ alkyl.

Embodiment 15. The compound of Embodiment 11, or a pharmaceutically acceptable salt thereof, wherein R⁷ and/or R¹⁰ is C₁-C₃ alkyl.

Embodiment 16 The compound of any one of Embodiments 6-8, or a pharmaceutically acceptable salt thereof, wherein y is 2 or 3 and R is halo.

Embodiment 17. The compound of Embodiment 9, or a pharmaceutically acceptable salt thereof, wherein y is 2 or 3 and R⁶ is halo.

Embodiment 18. The compound of Embodiment 10, or a pharmaceutically acceptable salt thereof, wherein y is 2 or 3 and R⁶ is halo.

Embodiment 19. The compound of Embodiment 11, or a pharmaceutically acceptable salt thereof, wherein y is 2 or 3 and R⁶ is halo.

Embodiment 20. The compound of Embodiment 12, or a pharmaceutically acceptable salt thereof, wherein y is 2 or 3 and R⁶ is halo.

Embodiment 21. The compound of any one of Embodiments 6-8, or a pharmaceutically acceptable salt thereof, wherein y is 1 and R⁸ is H.

Embodiment 22. The compound of Embodiment 9, or a pharmaceutically acceptable salt thereof, wherein y is 1 and R⁸ is H.

Embodiment 23. The compound of Embodiment 10, or a pharmaceutically acceptable salt thereof, wherein y is 1 and R³ is H.

Embodiment 24. The compound of Embodiment 11, or a pharmaceutically acceptable salt thereof, wherein y is 1 and R⁸ is H.

Embodiment 25. The compound of Embodiment 12, or a pharmaceutically acceptable salt thereof, wherein y is 1 and R⁸ is H.

Embodiment 26. The compound of Embodiment 13, or a pharmaceutically acceptable salt thereof, wherein y is 1 and R⁸ is H.

Embodiment 27. The compound of any one of Embodiments 6-8, or a pharmaceutically acceptable salt thereof, wherein R⁹ is H.

Embodiment 28. The compound of Embodiment 9, or a pharmaceutically acceptable salt thereof, wherein R⁹ is H.

Embodiment 29. The compound of Embodiment 10, or a pharmaceutically acceptable salt thereof, wherein R⁹ is H.

Embodiment 30. The compound of Embodiment 11, or a pharmaceutically acceptable salt thereof, wherein R⁹ is H.

Embodiment 31. The compound of Embodiment 12, or a pharmaceutically acceptable salt thereof, wherein R⁹ is H.

Embodiment 32. The compound of Embodiment 13, or a pharmaceutically acceptable salt thereof, wherein R⁹ is H.

Embodiment 33. The compound of Embodiment 14, or a pharmaceutically acceptable salt thereof, wherein R⁹ is H.

Embodiment 34. The compound of any preceding Embodiment, wherein the compound is of the formula

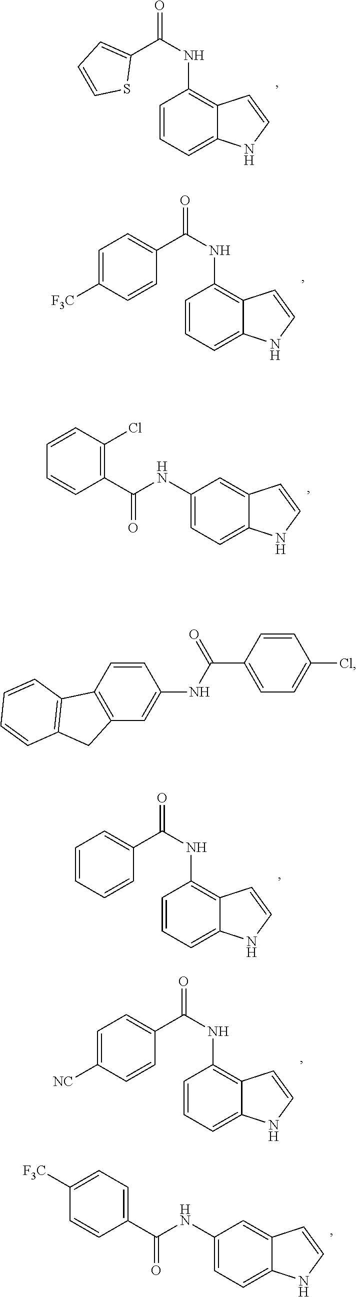

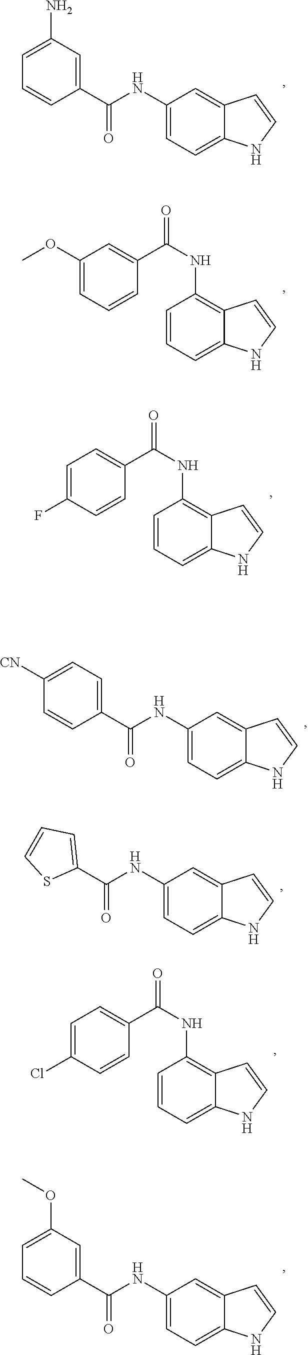

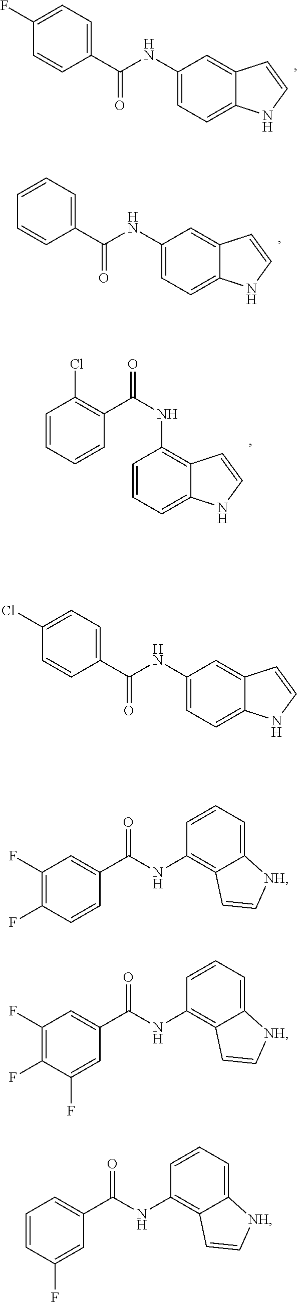

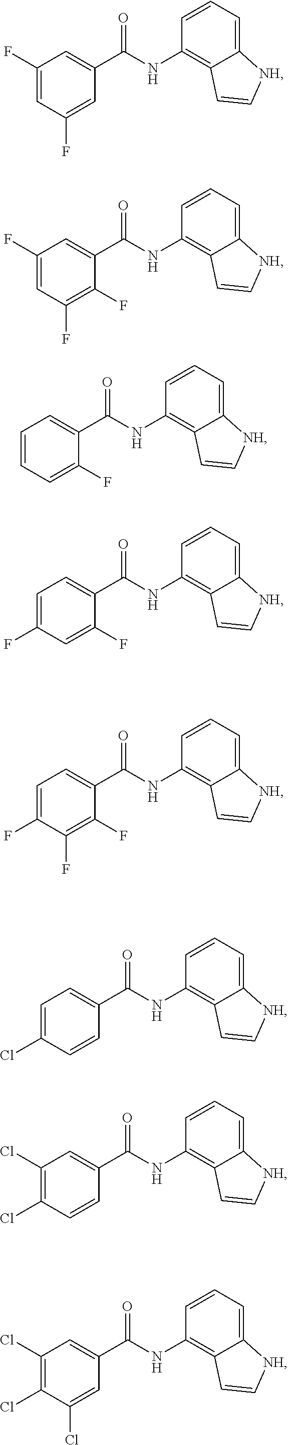

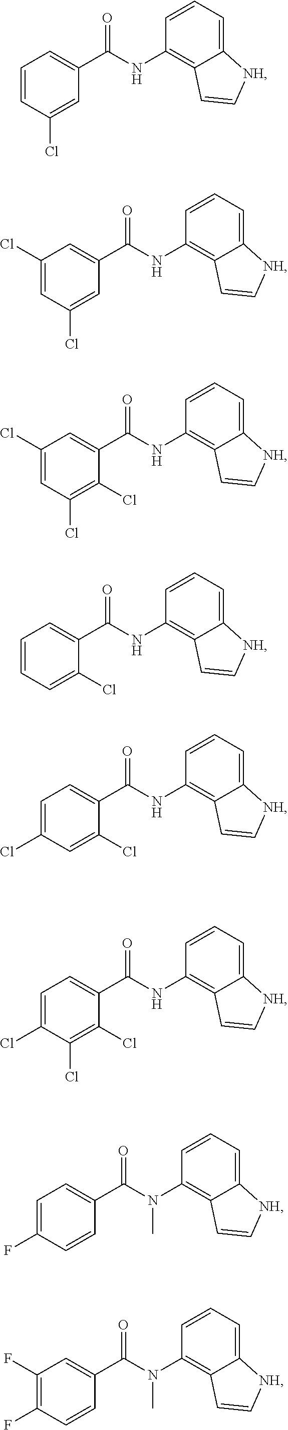

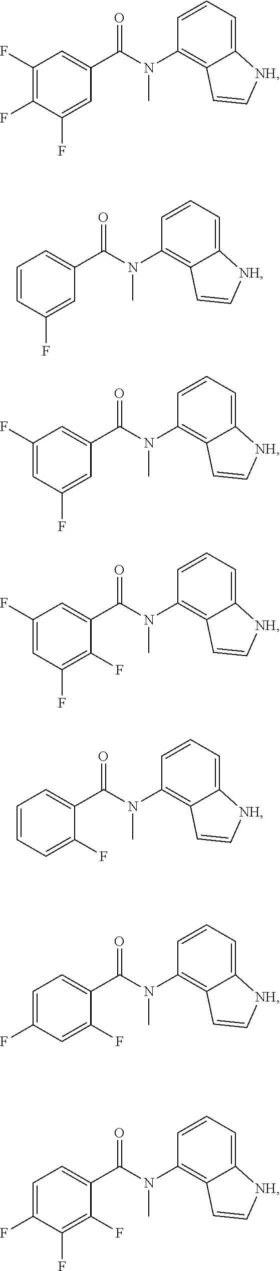

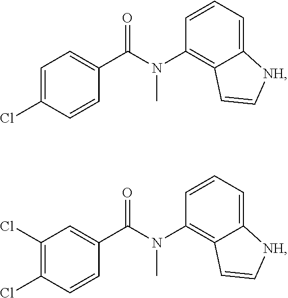 [SMILES: CN(C(=O)c1ccc(Cl)cc1)c1cccc2[nH]ccc12 ; CN(C(=O)c1ccc(Cl)c(Cl)c1)c1cccc2[nH]ccc12]

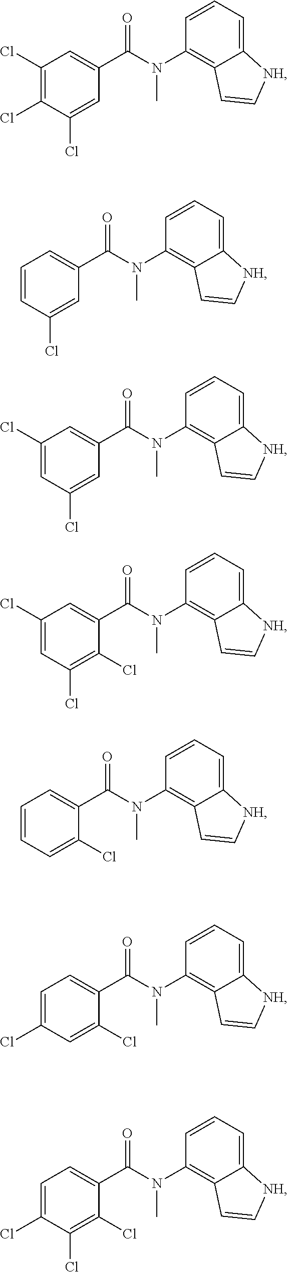

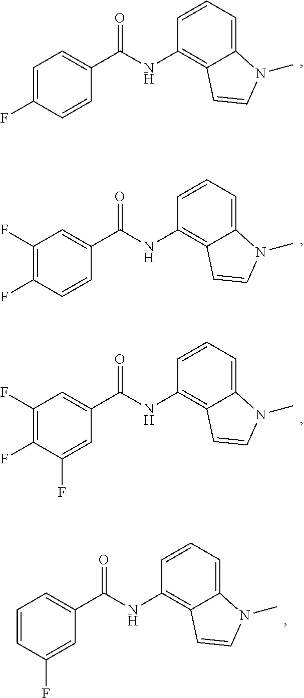 [SMILES: Cn1ccc2c(NC(=O)c3ccc(F)cc3)cccc21 ; Cn1ccc2c(NC(=O)c3ccc(F)c(F)c3)cccc21 ; Cn1ccc2c(NC(=O)c3cc(F)c(F)c(F)c3)cccc21 ; Cn1ccc2c(NC(=O)c3cccc(F)c3)cccc21]

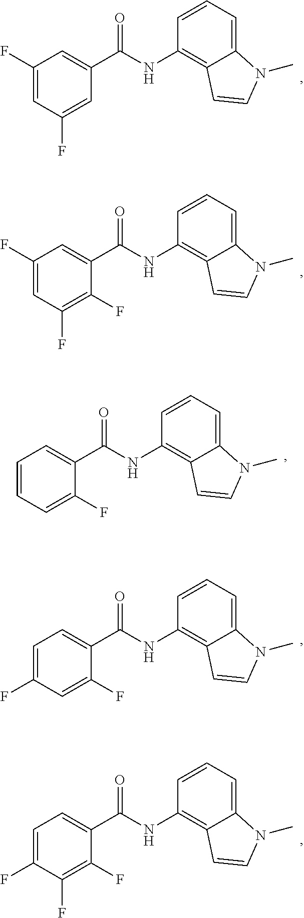 [SMILES: Cn1ccc2c(NC(=O)c3cc(F)cc(F)c3)cccc21 ; Cn1ccc2c(NC(=O)c3cc(F)cc(F)c3F)cccc21 ; Cn1ccc2c(NC(=O)c3ccccc3F)cccc21 ; Cn1ccc2c(NC(=O)c3ccc(F)cc3F)cccc21 ; Cn1ccc2c(NC(=O)c3ccc(F)c(F)c3F)cccc21]

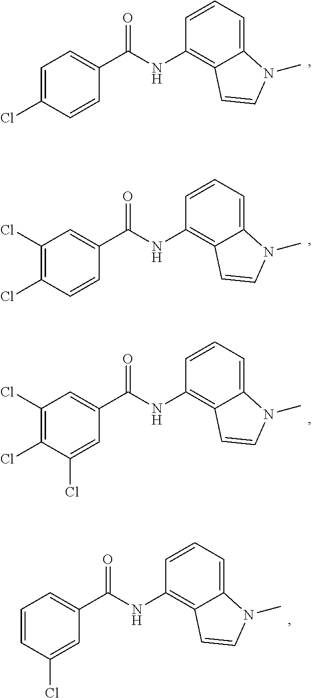 [SMILES: Cn1ccc2c(NC(=O)c3ccc(Cl)cc3)cccc21 ; Cn1ccc2c(NC(=O)c3ccc(Cl)c(Cl)c3)cccc21 ; Cn1ccc2c(NC(=O)c3cc(Cl)c(Cl)c(Cl)c3)cccc21 ; Cn1ccc2c(NC(=O)c3cccc(Cl)c3)cccc21]

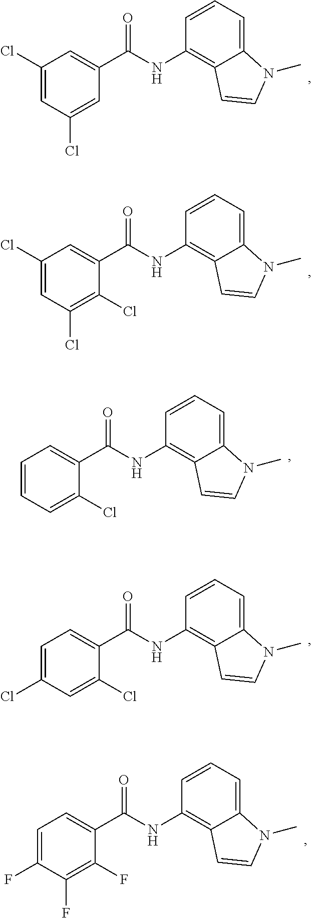 [SMILES: Cn1ccc2c(NC(=O)c3cc(Cl)cc(Cl)c3)cccc21 ; Cn1ccc2c(NC(=O)c3cc(Cl)cc(Cl)c3Cl)cccc21 ; Cn1ccc2c(NC(=O)c3ccccc3Cl)cccc21 ; Cn1ccc2c(NC(=O)c3ccc(Cl)cc3Cl)cccc21 ; Cn1ccc2c(NC(=O)c3ccc(F)c(F)c3F)cccc21]

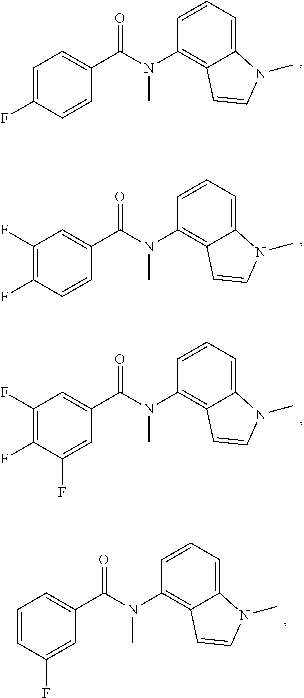 [SMILES: CN(C(=O)c1ccc(F)cc1)c1cccc2c1ccn2C ; CN(C(=O)c1ccc(F)c(F)c1)c1cccc2c1ccn2C ; CN(C(=O)c1cc(F)c(F)c(F)c1)c1cccc2c1ccn2C ; CN(C(=O)c1cccc(F)c1)c1cccc2c1ccn2C]

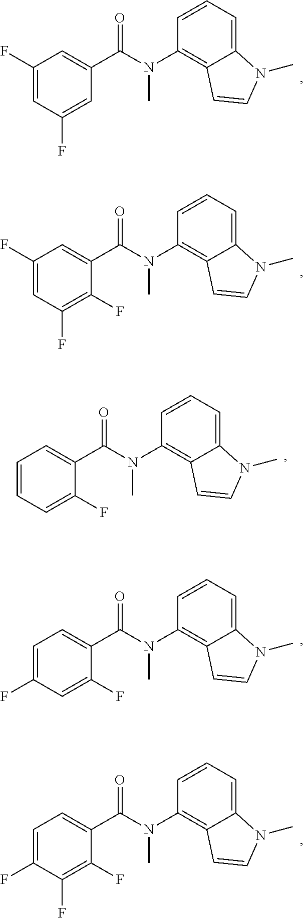 [SMILES: CN(C(=O)c1cc(F)cc(F)c1)c1cccc2c1ccn2C ; CN(C(=O)c1cc(F)cc(F)c1F)c1cccc2c1ccn2C ; CN(C(=O)c1ccccc1F)c1cccc2c1ccn2C ; CN(C(=O)c1ccc(F)cc1F)c1cccc2c1ccn2C ; CN(C(=O)c1ccc(F)c(F)c1F)c1cccc2c1ccn2C]

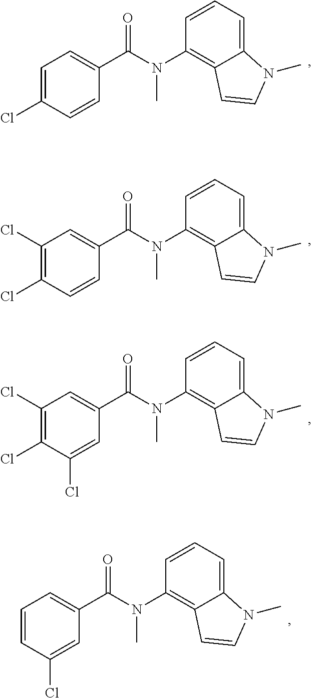 [SMILES: CN(C(=O)c1ccc(Cl)cc1)c1cccc2c1ccn2C ; CN(C(=O)c1ccc(Cl)c(Cl)c1)c1cccc2c1ccn2C ; CN(C(=O)c1cc(Cl)c(Cl)c(Cl)c1)c1cccc2c1ccn2C ; CN(C(=O)c1cccc(Cl)c1)c1cccc2c1ccn2C]

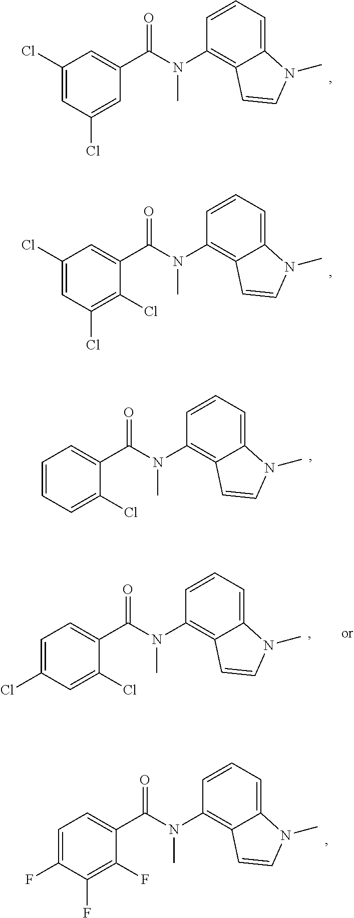 [SMILES: CN(C(=O)c1cc(Cl)cc(Cl)c1)c1cccc2c1ccn2C ; CN(C(=O)c1cc(Cl)cc(Cl)c1Cl)c1cccc2c1ccn2C ; CN(C(=O)c1ccccc1Cl)c1cccc2c1ccn2C ; CN(C(=O)c1ccc(Cl)cc1Cl)c1cccc2c1ccn2C ; CN(C(=O)c1ccc(F)c(F)c1F)c1cccc2c1ccn2C]

or a pharmaceutically acceptable salt thereof.

Embodiment 35. A pharmaceutical composition comprising the compound of any one of Embodiments 1-34, or a pharmaceutically acceptable salt thereof-, and a pharmaceutically acceptable carrier.

Embodiment 36. A method of inhibiting tubulin-associated unit (tau) protein aggregation in a subject having, or at risk for, tau protein aggregation, which method comprises administering to the subject the composition of Embodiment 34 in an amount effective to inhibit tau protein aggregation, whereupon tau protein aggregation is inhibited in the subject having, or at risk for, tau protein aggregation.

Embodiment 37. The method of Embodiment 36, wherein the subject has, or is at risk for, Alzheimer's disease.

Embodiment 38. A method of inhibiting alpha-synuclein (α-syn) protein aggregation in a subject having, or at risk for, α-syn protein aggregation, which method comprises administering to the subject the composition of Embodiment 35 in an amount effective to inhibit α-syn protein aggregation, whereupon α-syn aggregation is inhibited in the subject having, or at risk for, α-syn protein aggregation.

Embodiment 39. The method of Embodiment 38, wherein the subject has, or is at risk for, Parkinson's disease.

### EXAMPLES

The following examples serve to illustrate the present disclosure. The examples are not intended to limit the scope of the claimed invention in any way.

### Example 1

### Synthesis

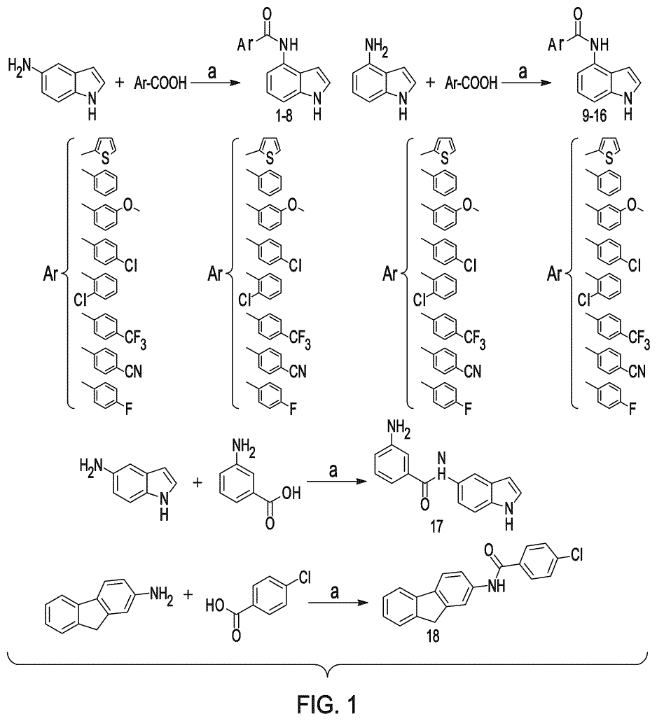

A series of seventeen 4- or 5-aminoindole carboxamides were synthesized using various commercially available substituted aromatic carboxylic acids employing the acid-amine coupling protocol. Indole carboxamide derivatives 1-17 were synthesized starting from 4- or 5-aminoindoles and commercially available carboxylic acids using the acid-amino coupling protocol, employing 1-ethyl-3-(3′-dimethylaminopropyl) carbodiimide·HCl (EDCI·HCl) and 4-(dimethylamino) pyridine in N, N-dimethylformamide (DMF) at 0-25° C. (FIG. 1). Compound 18 was prepared using the same procedure starting from 2-aminofluorene and 4-chlorobenzoic acid. All final compounds were obtained in moderate to good yields (49-76%) and characterized by 1H and 13C nuclear magnetic resonance (NMR) spectroscopy, high resolution mass spectrometry (HRMS), and melting point (MP).

All reagents and solvents were commercially available (Sigma Aldrich, St. Louis, MO; Thermo Scientific (formerly Alfa Aesar), Waltham, MA; Matrix Scientific, Columbia, SC; Ambeed, Arlington Hts, IL). Reagents were used without further purification. The progress of all reactions was monitored using TLC on precoated silica gel plates with fluorescent indicator detectable at 254 nm from Sigma Aldrich, Organic solutions were dried over anhydrous sodium sulfate. Evaporation of the solvents was carried out on a Büchi rotavapor R-100 equipped with a Büchi V-100 vacuum controller. Proton and carbon nuclear magnetic resonance (NMR) spectra were recorded on a Bruker 500 MHz spectrometer. Proton chemical shifts are reported in ppm (TMS, δ 0.00) or with the solvent reference relative to IMS employed as the internal standard (CDCl₃, δ 7.26; DMSO-d₆ δ 2.54). The multiplicities of NMR signals are designated as s (singlet), d (doublet), dd (double doublet), t (triplet), q (quartet), br (broad), in (multiplet, for unresolved lines). High resolution mass spectrometry (HRMS) of the compounds were carried out on Advion Mass Spectrometer (Advion Expression CMS) at the Analytical Mass Spectrometry Facility within the Purdue Institute for Drug Discovery. Uncorrected melting points (mp) were determined on a Barnstead Electrothermal Mel-Temp apparatus (Barnstead international, Dubuque, Iowa, USA). The physical chemical properties of each molecule were predicted using the following programs: SwissADME (TPSA, Log P, Log D), Blood-Brain Barrier Prediction Server (https://www.cbligand.org/BBB/), and Chemaxon (pKa) (Table 1).

### General Procedure for the Synthesis of Indole Carboxamides (1-17):

4-dimethylamino pyridine (1.1 eq) and N-(3-Dimethylaminopropyl)-N′-ethyl carbodiimide hydrochloride (1 eq) was added slowly to a stirred solution of appropriate carboxylic acid (1 eq) in anhydrous dimethylformamide (4 mL) at 0° C. The reaction was then allowed to stir for 45 minutes at 0° C. After this, 4- or 5-aminoindole (1.1 eq) was added and the reaction was allowed to warm to room temperature and stirred overnight. On completion, an aqueous solution of 1N hydrochloric acid (7 mL) and EtOAc (10 mL) was added to the reaction mixture and extracted. The organic layers were then washed with saturated solutions of ammonium chloride (7 mL), sodium bicarbonate (7 mL), and brine (7 mL), respectively. Organic layers were collected, filtered over anhydrous sodium sulfate, and dried in-vacuo to obtain the desired products with moderate to good yields.

Procedure for the synthesis of 4-chloro-N-(9H-fluoren-2-yl)benzamide (18):

To a stirred solution of 4-chlorobenzoic acid (100 mg, 0.64 mmol) in anhydrous dimethylformamide (4 mL) at 0° C., 4-dimethylamino pyridine (86 mg, 0.70 mmol) and V-(3-Dimethylaminopropyl)-N′-ethyl carbodiimide hydrochloride (122 mg, 0.64 mmol) was added slowly. The reaction was then allowed to stir for 45 minutes at 0° C. After this, 2-aminofluorene (127 mg, 0.70 mmol) was added and the reaction was allowed to warm to room temperature and stirred overnight. On completion, work-up was performed as described in 5.1.1 and resulted in pure product 18 (89 mg, 56%) as a yellow solid.

5.1.2.1. N-(1H-indol-4-ylthiophene-2-carboxamide (1). 134 mg, 71%; black solid; ¹H NMR (500 MHz, DMSO) δ 11.14 (s, 1H), 10.09 (s, 1H), 8.10 (dd, J=3.8, 1.2 Hz, 1H), 7.82 (dd, J=5.0, 11 Hz, 1H), 7.33-7.29 (m, 1H), 7.29-7.19 (m, 3H), 7.07 (t, J=7.8 Hz, 1H), 6.57-6.52 (m, 1H). ¹³C NMR (126 MHz, DMSO) δ 160.3, 140.8, 137.3, 131.9, 130.1, 129.5, 128.5, 125.0, 122.9, 121.4, 114.1, 109.2, 100.4. HRMS-ESI (m/z): [M+Na]⁺ calcd for C₁₃H₁₁N₂NaOS, 265.0412, found [M+Na]⁺ 265.1062; Melting Point: 185.9-188.2° C.

5.1.2.2. N-(1H-indol-4-yl)benzamide (2). 144 mg, 75%, black solid; ¹H NMR (500 MHz, DMSO) δ 11.12 (s, 1H), 10.06 (s, 1H), 8.00 (d, J=7.0 Hz, 2H), 7.61-7.55 (m, 1H), 7.52 (ddt, J=8.4, 6.7, 1.4 Hz, 2H), 7.37 (d, J=7.5 Hz, 1H), 7.29 (dd, J=3.1, 2.4 Hz, 1H), 7.24 (dt, J=8.1, 1.0 Hz, 1H), 7.07 (t, J=7.8 Hz, 1H), 6.59 (td, J=2.1, 1.0 Hz, 1H). ¹³C NMR (126 MHz, DMSO) δ 166 0, 137.3, 135.7, 131.8, 130.8, 128.8, 128.3, 124.8, 122.7, 121.3, 113.6, 109.0, 100.4. HRMS-ESI (m/z) [M+H]⁺ calcd for C₁₅H₁₃N₂O, 237.1028, found [M+H]⁺ 237.0736; Melting Point: 181.9-186.9° C.

5.1.2.3. N-(1H-indol-4-yl)-3-methoxybenzanilide (3). 103 mg, 59%, dark purple solid; ¹H NMR (500 MHz, DMSO) δ 11.12 (s, 1H), 10.06 (s, 1H), 7.57 (dd, 1=22.2, 5.1 Hz, 2H), 7.43 (t, 79 Hz, 1H), 7.38-7.21 (m, 3), 7.14 (dd, J=8.2, 2.6 Hz, 1H), 7.07 (t, J=7.8 Hz, 1H), 6.57 (t, J=2.5 Hz, 1H), 3.84 (s, 3H). ¹³C NMR (126 MHz, DMSO) δ 165.7, 159.6, 137.3, 137.0, 130.7, 129.9, 124.8, 122.8, 121.3, 120.5, 117.7, 113.8, 113.4, 109.0, 100.5, 55.8. HRMS-ESI (m/z): [M+H]⁺ calcd for C₁₆H₁₅N₂O₂, 267.1134, found [M+H]⁺ 267.1211; Melting Point: 196.9-199.0° C.

5.1.2.4. 4-chloro-N-(1H-indol-4-yl)benzamide (4). 110 mg, 64%, black solid; ¹H NMR (500 MHz, DMSO) δ 11.13 (s, 1H), 10.15 (s, 1H), 8.02 (d, J=8.6 Hz, 2H), 7.59 (d, J=8.5 Hz, 2H), 7.35 (d, J=7.6 Hz, 1H), 7.30 (dd, J=3.1, 2.4 Hz, 1H), 7.24 (dt, J=8.0, 0.9 Hz, 1H), 7.07 (t, J=7.8 Hz, 1H), 6.57 (s, 1H). ¹³C NMR (126 MHz, DMSO) δ 164.9, 137.3, 136.6, 134.4, 130.5, 130.3, 128.9, 124.9, 122.7, 121.3, 113.7, 109.1, 100.4. HRMS-ESI (m/z): [M+H]⁺ calcd for C₁₅H₁₂ClN₂O, 271.0638, found [M+H]⁺ 271.11601 Melting Point: 170.9-175.4° C.

5.1.2.5. 2-chloro-N-(1H-indol-4-yl)benzamide (5). 88 mg, 51%, dark purple solid; ¹H NMR (500 MHz, DMSO) δ 11.13 (s, 1H), 10.25 (s, 1H), 7.68-7.38 (m, 5H), 7.28 (t, J=2.8 Hz, 1H), 7.22 (d, J=8.0 Hz, 1H), 7.07 (t, J=7.9 Hz, 1H), 6.72 (t, J=2.6 Hz, 1H). ¹³C NMR (126 MHz, DMSO) δ 165.5, 137.9, 137.3, 131.3, 130.6, 130.5, 130.0, 129.6, 127.6, 124.8, 121.6, 121.4, 112.1, 108.8, 100.2. HRMS-ESI (m/z): [M+H]⁺ calcd for C₁₅H₁₂ClN₂O, 271.0638, found [M+H]⁺ 271.1155; Melting Point: 193.7-197.8° C.

5.1.2.6. N-(1H-indol-4-yl)-4-(rifluoromethyl)benzamide (6). 85 mg, 53%, dark violet solid; ¹H NMR (500 MHz, DMSO) δ 11.15 (s, 1H), 10.30 (s, 1H), 8.18 (d, J=8.0 Hz, 2H), 7.90 (d, J=8.1 Hz, 2H), 7.38 (d, J=7.6 Hz, 1H), 7.35-7.28 (m, 1H), 7.25 (dt, J=8.1, 0.9 Hz, 1H), 7.08 (t, J=7.8 Hz, 1H), 6.59 (t, J=1.1 Hz, 1H). ¹³C NMR (126 MHz, DMSO) δ 165.0, 139.5, 137.3, 130.3, 129.2, 125.8, 125.8, 125.0, 123.4, 122.6, 121.3, 113.7, 109.3, 100.4. HRMS-ESI (m/z): [M+H]⁺ calcd for C₁₆H₁₂F₃N₂O, 305.0902. found [M+H]⁺ 305.1576; Melting Point: 209.0-211.6° C.

5.1.2.7. 4-cyano-N-(1H-indol-4-yl)benzamide (7). 113 rug, 63%, ash colored solid; ¹H NMR (500 MHz, DMSO) δ 11.17 (s, 1H), 10.34 (s, 1H), 8.09-8.04 (m, 1H), 8.04-7.99 (m, 2H), 7.97 (d, J=8.6 Hz, 1H), 7.37 (d, J=7.6 Hz, 1H), 7.31 (dd, J=3.1, 2.4 Hz, 1H), 7.25 (dt, J=8.1, 0.9 Hz, 1H), 7.07 (t, J=7.9 Hz, 1H), 6.57 (s, 1H). ¹³C NMR (126 MHz, DMSO) δ 164.7, 139.7, 137.3, 132.9, 130.4, 129.2, 125.0, 122.6, 121.3, 118.9, 114.1, 113.7, 109.3, 100.4. HRMS-ESI (m/z): [M+H]⁺ calcd for C₁₆H₁₂N₃O, 262.0980, found [M+H]⁺ 262.9301; Melting Point: 200.4-207.1° C.

5.1.2.8. 4-fluoro-N-(1H-indol-4-yl)benzamide (8). 68 mg, 75%, ash colored solid; ¹H NMR (500 MHz, DMSO) δ 11.13 (s, 1H), 10.09 (s, 1H), 8.16-8.02 (m, 2H), 7.43-7.28 (m, 41H), 7.24 (d, J=8.1 Hz, 1H), 7.07 (t, J=7.8 Hz, 1H), 6.66-6.50 (in, 1H). ¹³C NMR (126 MHz, DMSO) δ 164.9, 163.5, 137.3, 132.1, 131.1, 131.0, 130.6, 124.9, 122.8, 121.4, 115.8, 115.6, 113.8, 109.1, 100.4. HRMS-ESI (m/z): [M+H]⁺ calcd for C₁₅H₁₂FN₂O, 255.0934, found [M+H]⁺ 255.0931. Melting Point: 161.3-162.5° C.

5.1.2.9. N-(1H-indol-5-yl)thiophene-2-carboxamide (9). 113 mg, 60%, beige colored solid; ¹H NMR (500 MHz, DMSO) δ 11.04 (s, 1H), 10.06 (s, 1H), 8.00 (d, J=3.8 Hz, 1H), 7.96-7.86 (m, 1H), 7.79 (dd, J=5.0, 1.1 Hz, 1H), 7.39-7.29 (m, 3H), 7.20 (dd, J=5.0, 3.7 Hz, 1H), 6.40 (s, 1H). ¹³C NMR (126 MHz, DMSO) δ 160.1, 141.3, 133.5, 131.6, 130.8, 128.9, 128.4, 127.9, 126.5, 116.6, 112.9, 111.6, 101.6; HRMS-ESI (m/z): [M+Na]⁺ calcd for C₁₃H₁₁N₂NaOS, 265.0412, found [M+Na], 265.1062, Melting Point: 176.4-179.9° C.

5.1.2.10. N-(1H-indol-5-yl)benzamide (10). 144 mg, 67%, off white solid; ¹H NMR (500 MHz, DMSO) δ 11.02 (s, 1H), 10.07 (s, 1H, 8.04-7.89 (m, 3H), 7.59-7.46 (m, 3H), 7.43-7.25 (m, 3H), 6.40 (s, 1H). ¹³C NMR (126 MHz, DMSO) δ 165.6, 135.9, 133.5, 131.6, 131.4, 128.8, 128.0, 127.9, 126.4, 116.6, 112.7, 111.5, 101.6. HRMS-ESI (m/z): [M+H]⁺ calcd for C₁₅H₁₃N₂O, 237.1028, found [M+H]⁺ 237.1093; Melting Point: 168.0-170.0° C.

5.1.2.11. N-(1H-indol-5-yl)-3-methoxybenzamide (11). 100 mg, 57%, beige colored solid ¹H NMR (500 MHz, DMSO) δ 11.02 (s, 1H), 10.04 (s, 1H), 7.97 (d, J=1.9 Hz, 1H), 7.59-7.52 (m, 1H), 7.50 (dd, J=2.6, 1.6 Hz, 1H), 7.45-7.28 (m, 4H), 7.12 (ddd, J=8.2, 2.7, 1.0 Hz, 11H), 6.40 (d, J=1.0 Hz, 1H), 3.83 (s, 3H). ¹³C NMR (126 MHz, DMSO) δ 165.3, 159.6, 137.3, 133.5, 131.3, 129.9, 127.9, 126.4, 120.3, 117.5, 116.7, 113.2, 112.8, 111.5, 101.6, 55.8. HRMS-ESI (m/z): [M+H]⁺ calcd for C₁₆H₁₅N₂O₂, 267.1134, found [M+H]⁺ 267.1214; Melting Point: 157.9-161.7°.

5.1.2.12. 4-chloro-N-(1H-indol-5-yl)benzamide (12). 127 mg, 73%, beige colored solid; ¹H NMR (500 MHz, DMSO) δ 11.03 (s, 1H), 10.14 (s, 1H), 8.03-7.95 (m, 3H), 7.58 (d, J=8.5 Hz, 2H), 7.40-7.33 (m, 2H), 7.32 (t, J=2.7 Hz, 1H), 6.57-6.23 (m, 1H). ¹³C NMR (126 MHz, DMSO) δ 164.4, 136.5, 134.6, 133.5, 131.2, 130.0, 128.9, 127.9, 126.4, 116.6, 112.8, 111.5, 101.7. HRMS-ESI (m/z): [M+H]⁺ calcd for C₁₅H₁₂ClN₂O, 271.0638, found [M+H]⁺ 271.1162; Melting Point: 212.4-214.9° C.

5.1.2.13. 2-chloro-N-(1H-indol-5-yl)benzamide (13). 96 mg, 56%, beige colored solid; ¹H NMR (500 MHz, DMSO) δ 11.03 (s, 1H), 10.26 (s, 1H), 8.00 (s, 1H), 7.55 (ddd, J=11.8, 7.6, 1.7 Hz, 21H), 7.51-7.39 (m, 2H), 7.36-7.28 (m, 3H), 6.45-6.34 (m, 1H). ¹³C NMR (126 MHz, DMSO) δ 164.9, 138.0, 133.4, 131.4, 131.2, 130.5, 130.1, 129.4, 127.9, 127.7, 126.5, 115.6, 111.6, 101.7. HRMS-ESI (m/z): [M+H]⁺ calcd for C₁₅H₁₂ClN₂O, 271.0638, found [M+H]⁺ 271.1165; Melting Point: 221.7-230.3° C.

5.1.2.14. N-(1H-indol-4-yl)-4-(triiodomethyl)benzamide (14) 121 mg, 76%, beige colored solid; ¹H NMR (500 MHz, DMSO) δ 11.05 (s, 1H), 10.30 (s, 1H), 8.16 (d, J=81 Hz, 2H), 8.00 (d, J=1.8 Hz, 1H), 7.89 (d, J=8.2 Hz, 2H), 7.47-7.27 (m, 3H), 6.48-6.34 (m, 1H). ¹³C NMR (126 MHz, DMSO) δ 164.4, 139.8, 133.6, 131.1, 128.9, 127.9, 126.5, 125.8, 125.8, 123.4, 116.5, 112.8, 111.6, 101.7. HRMS-ESI (m/z): [M+Na]⁺ calcd for C₁₆H₁₂F₃N₂NaO, 327.0721, found [M+Na]⁺ 327.1426; Melting Point: 202.6-209.9° C.

5.1.2.15. 4-cyano-N-(1H-indol-5-yl)benzamide (15). 122 mg, 69%, off white solid; ¹H NMR (500 MHz, DMSO) δ 11.06 (s, 1H), 10.31 (s, 1H), 8.11 (d, J=8.3 Hz, 2H), 8.00 (d, J=8.5 Hz, 3H), 7.43-7.28 (m, 31H), 6.41 (s, 1H). ¹³C NMR (126 MHz, DMSO) δ 164.1, 140.0, 133.7, 133.1, 132.9, 131.0, 130.4, 128.9, 127.9, 126.5, 118.9, 116.5, 114.0, 112.8, 111.6, 101.7. HRMS-ESI (m/z): [M+H]⁺ calcd for C₁₆H₁₂N₃O, 262.0980, found [M+H]⁺ 262.0978; Melting Point: 212.0-216.6° C.

5.1.2.16. 4-fluoro-N-(1H-indol-5-yl)benzamide (16) 118 mg, 65%, beige colored solid; ¹H NMR (500 MHz, DMSO) δ 11.03 (s, 1H), 10.09 (s, 1H), 8.04 (dd, J=8.7, 5.6 Hz, 2H), 8.01-7.91 (m, 1H), 7.44-7.23 (m, 5H), 6.40 (d, J=2.5 Hz, 1H). ¹³C NMR (126 MHz, DMSO) δ 164.5, 133.5, 132.3, 132.3, 131.3, 130.7, 130.6, 127.9, 126.4, 116.6, 115.8, 115.6, 112.8, 111.5, 101.6. HRMS-ESI (m/z): [M+H]⁺ calcd for C₁₅H₁₂FN₂O, 255.0934, found [M+H]⁺ 255.0936; Melting Point: 206.1-210.2° C.

5.1.2.17. 3-amino-(1H-indol-5-yl)benzamide (17). 89 mg, 49%. pale yellow oil; ¹H NMR (500 MHz, DMSO) δ 10.98 (s, 1H), 9.86 (s, 1H), 7.96 (s, 1H), 7.47-7.22 (n, 3H), 7.22-7.01 (m, 3H), 6.79-6.65 (m, 1H), 6.38 (s, 1H), 5.26 (s, 2H). ¹³C NMR (126 MHz, DMSO) δ 166.4, 149.2, 137.0, 133.3, 131.7, 129.2, 127.9, 126.3, 116.9, 116.5, 115.1, 113.50, 112.5, 111.4, 101.6. HRMS-ESI (m/z): [M+H]⁺ calcd for C₁₅H₁₄N₃O, 252.1137, found [M+H]⁺ 252.1135.

5.1.2.18. 4-chloro-N-(9H-fluoren-2-yl)benzamide (18). 89 ng 56%, yellow solid; ¹H NMR (500 MHz, DMSO) δ 10.38 (s, 1H), 8.08 (d, J=1.9 Hz, 1H), 8.00 (d, J=8.4 Hz, 2H), 7.83 (dd, J=14.0, 7.9 Hz, 2H), 7.73 (dd, J=8.2, 2.0 Hz, 1H), 7.61 (d, J=8.4 Hz, 2H), 7.54 (d, J=7.5 Hz, 1H), 7.35 (t, J=7.5 Hz, 1H), 7.26 (t, J=7.4 Hz, 1H), 3.92 (s, 2H). ¹³C NMR (126 MHz, DMSO) δ 164.8, 144.1, 143.4, 141.4, 138.5, 137.4, 136.8, 134.2, 130.1, 129.0, 127.2, 126.7, 125.5, 120.5, 120.0, 119.7, 117.7, 37.0. HRMS-ESI (m/z): [M+H]⁺ calcd for C₂₀H₁₅ClNO, 320.0842, found [M+H]⁺ 321 1317. Melting Point: 244.8-246.8° C.

For each carboxamide compound, the physicochemical parameters were predicted using SwissADME. Blood-Brain Barrier (BBB) Prediction Server (https://www[dot]cbligand[dot]org/BBB), and Chemaxon. The chemical structures and physicochemical parameters are indicated in Table 1. During the design of the molecules that target prone-to-aggregate neuropeptides, the physicochemical properties relevant to cross the BBB were considered. The favorable physicochemical properties for ideal molecules to cross the BBB consist of the following: a MW≤397.9, a calculated partition coefficient (c log P) ranging from 1.7 and 3 (below ~2.8), and a topological polar surface area (TPSA) below 80-90 A°. These physicochemical median values are based on a Pfizer study, which has evaluated 227 central nervous system (CNS) drug candidates and set a CNS Multiparameter Optimization scoring (CNS MPO candidates), Among all the synthesized compounds, compounds 1, 2, 3, 7, 10, 11, 15 and 17 have a calculated log P below 2.8. All amide derivatives display a MW be; pw 397.9 and a TPSA value below 80-90 A°. On a scale of 6, the CNS MPO scores ranging from 3.5 to 4.5 are considered having potential to cross the BBB. Amide derivatives 2 to 4 and 7 to 17 exhibited a CNS MPO score withing this range. No compounds exhibited a higher score, which would have been considered more desirable.

### Example 2

### Effect of Compounds on Kinetics of α-Synuclein (α-Syn) Fibrillization as Monitored by Thioflavin-T (ThT) Fluorescence Assay

α-Syn was purchased from rPeptide, LLC (Watkinsville, GA). Briefly, human WIT aSYN and the was purified from BL21 DE3 E. coli cells. Protein expression was induced by the addition of isopropyl β-d-1-thiogalactopyranoside (IPTG) at 37° C. for 4 hours. Next, the cells were collected by centrifugation, and a lysate prepared form the cells was supplemented with ammonium sulfate to precipitate unwanted proteins via a salting out method. The supernatant containing aSYN was passed through a HiLoad 16/600 Superdex 200 size exclusion column, followed by a HiPrep Q HP 16/10 (Cytiva) anion-exchange column. Protein fractions enriched with aSYN (identified via SDS-PAGE with Coomassie blue staining) were pooled, and the solution was dialyzed against PBS (pH 7.4) buffer and stored at −20° C. until use. Concerning the non-phosphorylated tau 2N4R, bacterial expression plasmid consisting of the vector pRK172 carrying a cDNA encoding the human Tau 2N4R isoform was a kind gift of Dr. David Eliezer (Weill Cornell Medicine). For protein expression, E. coli B121 (DE3) cells were transformed with the plasmid and grown in LB media supplemented with ampicillin (100 μg/mL). Protein over-expression was induced by the addition of 1 mM IPTG for 4 h at 37° C., and cells were pelleted by centrifugation at 6,000 g for 15 min at 4° C. The cells were resuspended in lysis buffer (20 mM MES, 400 mM NaCl, 0.2 mM MgCl₂, 1 mM EGTA, protease inhibitor cocktail (P8340, Sigma Aldrich), 0.25 mg/mL lysozyme, and 1 pig/mL DNase I, pH 6.8) and lysed by a French press cell disruptor at 4° C., and the lysate was boiled for 20 minutes. Denatured proteins were pelleted by centrifugation at 30,000 g for 30 min at 4° C., and the supernatant was dialyzed overnight against cation exchange buffer (20 mM MES, 50 mM NaCl, 1 mM MgCl₂, 1 mM EGTA, 2 mM DTT, 0.1 mM PMSF, pH 6.8). The dialysate was loaded onto a HiPrep SP HP column, and proteins were eluted with a linear gradient ranging from 50 mM to 1 M NaCl. Fractions containing tau isoform 2N4R were pooled, and the resulting protein solution was dialyzed against PBS (pH 7.4) and stored at −80° C.

ThT fluorescence is routinely established assay to follow the kinetics of α-syn fibril formation with different drug candidate treatments [26, 27]. Compounds were first tested at a final of concentration of 100 μM and α-syn and 2N4R tau were prepared at 2 μM and 6 μM, respectively. Compounds with highest anti-fibrillary activity were tested at 3.125, 6.25, 12.5, 25, 50, and 100 μM to obtain dose-response curves.

For the α-syn ThT assay, ThT was used at a final concentration of 20 μM. α-Syn was dissolved in 20 mM Tris-HCl (pH 7.4) supplemented with 100 mM NaCl to a stock solution of 280 μM (1 mg/250 μL) prior to resuspension in ThT buffer to obtain a final concentration of 2 μM. Compounds and ThT were first added to the wells. The kinetics of fibril formation begin when the α-syn was solubilized in the ThT buffer (10 mM PBS buffer (pH 7.4), supplemented with 0.5 mM SDS and 300 mM NaCl) and added to a non-treated black 96 well microplate with a transparent flat bottom. Each well was filled with a maximum volume of 150 μL buffer with one 3 mm borosilicate bead [33]. The background fluorescence signal consisted of ThT in buffer and 0.25% DMSO without α-syn. The excitation and emission wavelengths were set at 440 and 485 nm, respectively, with a Synergy HT multi-mode microplate reader (BioTek, Winooski, VT). Measurements were taken at 37° C. every 20 minutes over 50 hours of shaking prior to reading the plate. Samples were measured in three replicates and the experiments were repeated three times using different α-syn stock solutions. For each time point, the background fluorescence signal was subtracted. Arbitrary units of fluorescence were calculated from the mean values normalized against the maximum value in each completed assay. Arbitrarily, the maximum value (100%) for the fluorescence intensity was established for α-syn.

Concerning the non-phosphorylated tau (isoform 2N4R) kinetics of fibril formation, ThT was performed with the tau protein solution was diluted to a final concentration of 6 μM in PBS (pH 7.4) supplemented with 1.5 μM heparin, 20 μM ThT, 2.5 mM DTT, and 100 μM compound. Aliquots of the diluted protein solution (100 μL each) were pipetted into the wells of a 96-well plate, and a Teflon ball was added to each well. The plate was incubated at 37° C. with constant shaking at 1,000 rpm in a Tecan Stark plate reader. ThT fluorescence was measured every 15 min with excitation and emission wavelengths of 440 nm and 480 nm, and the data were plotted using GraphPad Prism.

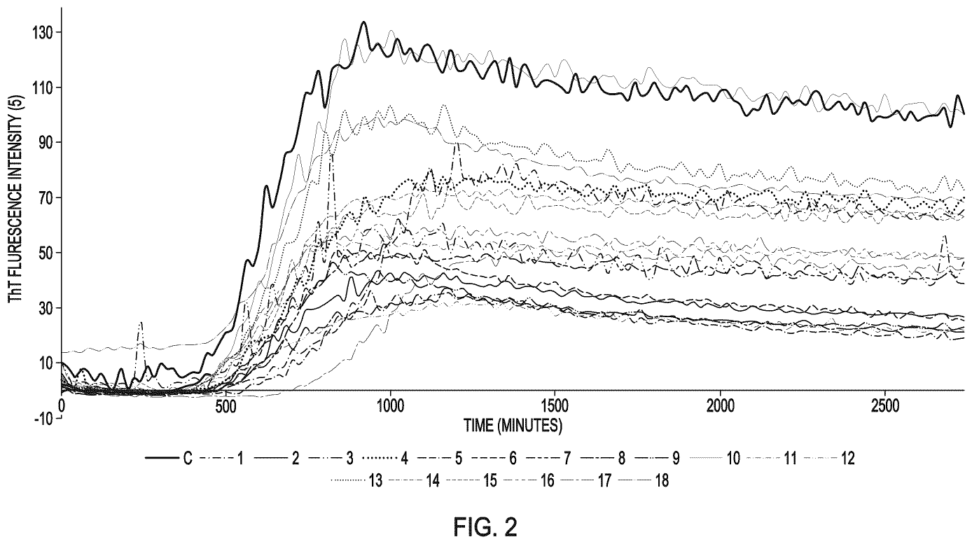

ThT fluorescence assay using alpha-synuclein (α-syn) was employed to identify the most potent anti-fibrillar compounds among the amide series. Compounds were tested at 100 μM with α-syn at 6 μM, resulting in a molar ratio of approximately 1:16 (protein:compound). The complete kinetics of fibril formation for these compounds are presented in FIG. 2. The plateau phase of sigmoidal kinetics is of particular interest because it is the phase in which mature fibrils form and where aggregation and disaggregation are at equilibrium with each other; therefore, the fluorescence intensity between multiple compounds at the plateau phase can be compared to identify the compounds with the most promising anti-aggregation activity (Table 1, column α-syn ThT %). This initial ThT fluorescence assay identified compounds 2, 4, 5, 6, 7, and 8 as the most potent anti-fibrillary compounds, where fibrillation was reduced to 30% or less compared to the control condition (Table 1). Compound 17 was the best representative of the 5-aminoindole series and moved to the tier-2 assays, even though the resulting ThT percentage of fluorescence intensity was 10% higher than the cut off. These compounds were then tested on tau 2N4R ThT fluorescence assays.

In terms of structure-activity relationships, compound 18 was prepared to link prior anti-aggregation chemistry work with the 2-aminofluorene substituent [25, 26]. However, the aminoindole substituent has been identified as an interesting molecular component of urea derivatives to inhibit the α-syn oligomer and fibril formation [27]. Compound 18 was compared with a series of 4- or 5-aminoindole carboxamides. Based on the results obtained (Table 1), compound 18, which bears a 2-aminofluorene coupled with p-chlorobenzene acid, resulted in a ThT fluorescence intensity of 69.4%, confirming the effectiveness of the aminoindoles (compounds 4 and 12) in abrogating α-syn fibril formation. The 4-aminoindole carboxamides (compounds 1 to 8) were more potent compared to 5-aminoindole carboxamides (compounds 9 to 17). In the case of 5-aminoindole carboxamides, the anti-fibrillar activities were lower with ThT fluorescence intensities ranging between 40-102%, with compound 17 (40%) being the best 5-aminoindole representative.

Among the 4-aminoindole derivatives, compound 2 (27%), which bears an unsubstituted phenyl ring, was more potent than compound 1 (64%), which bears a heteroaromatic ring (thiophene). The thiophene group is known as an isostere of phenol, and this may indicate that the phenyl ring with a substituted electron donating group, such as hydroxyl, may provide the best anti-aggregation activities. When the methoxy is introduced on the aromatic right at the para position, the fluorescence intensity increases as seen in compound 3 (43%) when compared to unsubstituted carboxylic acids (compound 2, 27%). The Hammett equation classifies a methoxy substituent at the meta position as an electron-withdrawing group [28-30]. Substitutions with a more diverse selection of electron-withdrawing groups, such asp-chloro (compound 4; 22%), o-chloro (compound 5; 19.5%), p-trifluoromethyl (compound 6; 26.7%), cyano (compound 7; 22.9%), and p-fluoro (compound 8; 22%), confirmed the importance of such electronic modifications for anti-fibrillar activity with α-syn.

### Example 3

### Confirmation of Anti-Fibrillary Activity Using Tau Isoform 2N4R in ThT Fluorescence Assay

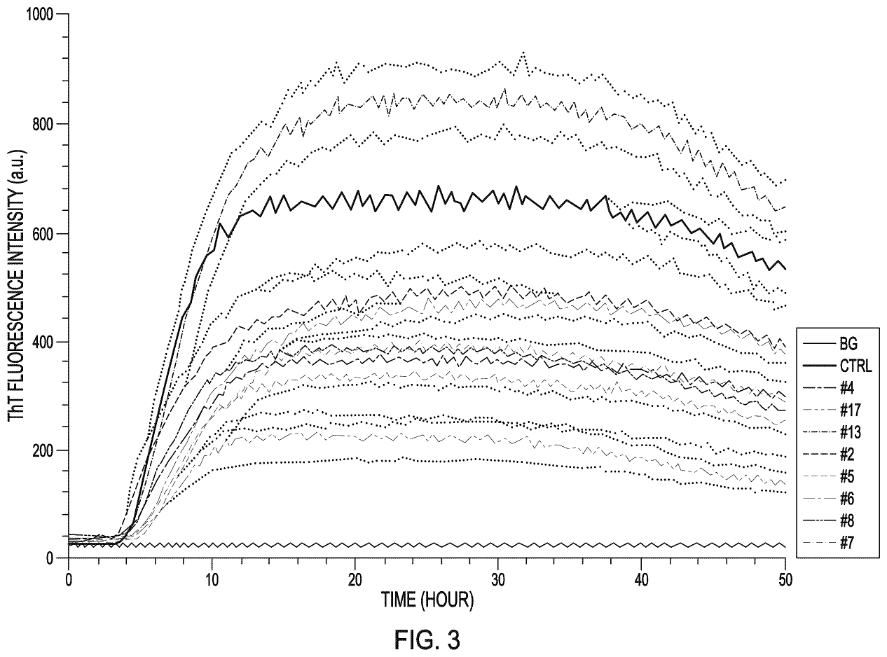

The inhibitory potential of the best amide representatives on tau isoform 2N4R fibril formation was investigated, and which derivatives exhibited a robust cytoprotective (and/or anti-inclusion) effect with cell-based assays was examined. Various structural analogs were first tested at a final concentration of 100 μM in the presence of tau 2N4R at 6 μM. Compounds 2, 4-8, and 17 were pre-selected with α-syn ThT assay prior to evaluation on the tau isoform 2N4R. The kinetics of aggregation curves resulting from the ThT experiment are shown in FIG. 3. Representative of the 4-aminoindole series, compounds 4, 5, 7, 8, resulted in good inhibitory effect, with compounds 2 and 6 exhibiting a weaker anti-fibrilization effect. Interestingly, the 5-aminoindole representative, compound 17, was the best inhibitor of tau isoform 2N4R fibril formation Compound 13 increased the fluorescence intensity, i.e., fibrillization, and was used as a negative control for most of the tier-2 assays.

### Example 4

Monitoring of α-Syn and Tau Oligomer Formation with Aminoindole Carboxamides by Photo-Inducing Cross Uncoupled Protein (PICUP) Assay

To force the oligomerization by cross-linking, α-syn (from Rpeptide, LLC) and tau isoform 2N4R were diluted in 10 mM phosphate buffer (pH 7.4) to reach a final concentration of 10 μM [27]. Different compounds were added to the protein solution at a final concentration of 50 μM, resulting in a molar ratio of 1:5. To confirm the gradual effect of the compounds on the inhibition of α-syn oligomerization, compounds were tested at final concentration of 3.125, 6.25, 12.5, 25, and 50 μM. The controls consisted of samples without light exposition, without Ru(bpy), and without compound (i.e., 0.125% DMSO). The cross-linking reaction was initiated by the addition of 2 μL of Ru(bpy) (300 μM final concentration) and 2 μL ammonium persulfate (6 mM final concentration). Samples were irradiated immediately. Light exposition was of 1 second duration for ca-syn and 3 second duration for tau isoform 2N4R, with a 53 W (120 V) incandescent lamp installed in a homemade dark-box. Each tube contained a final volume of 20 μL. After irradiation, 8.3 μL. of Lammeli loading buffer containing 15% β-mercaptoethanol was immediately added to the solution, followed by incubation at 95° C. for 10 minutes. The cross-linked samples were separated on a 16% SDS-Page gel and visualized by Coomassie blue staining.

To validate the PICUP results, the uncross linked α-syn (produced in Dr. Rochet lab) was incubated at a final concentration of 60 μM in 10 mM phosphate buffer (pH 7.4) with the presence of vehicle (1.5% DMSO), the amide compounds of interest (Compounds 2, 8, 17), and the weak anti-fibrillization inhibitor (compound 13). Compounds were tested at a final concentration of 600 μM (molar ratio 1:10). Samples were incubated for 24 h and 48 h with constant shaking 300 rpm at 37° C. (VWR Thermal Shake Touch, Troemner, LLC, Thorofare, NJ). After completion of the incubation time, samples were mixed with one third volume of above-mentioned loading buffer and separated by electrophoresis in a 16% SDS-Page gel. After transfer onto a nitrocellulose membrane, Western blot was achieved with previously published procedures [34, 35] to detect oligomers and α-syn using a polyclonal anti-oligo A11 (Invitrogen, Waltham, Massachusetts, cat. #AHB0052) and monoclonal anti-α-syn (Invitrogen, syn 211, cat. #32-8100), respectively. Nitrocellulose membranes were exposed to the monoclonal antibodies diluted at 1/1000 using a 5% non-fat dried milk with TBS and 0.1% Tween-20 (TBS-T) for 12 h at 4° C. Peroxidase conjugated secondary antibodies consisted of an anti-rabbit (Rockland Immunochemicals, inc., Pottstown, PA, cat. #611-1322-0100) and an anti-mouse (Rockland, cat. #610-1319-0100) utilized at a dilution of 1/5000 in 5% non-fat dried milk with TBS-T for one hour at room temperature. The membranes were then exposed to a 1:1 ratio of enhanced chemiluminescent solution (Thermo Scientific, Rockford, IL, cat. #32209) for one minute before the acquisition of images using a SynGene G;Box scanner (model Chemi XR 5 and the G:Box Chemi-XRQ GENESYS software.

One end point used to verify the propensity of misfolded protein in the drug discovery field is the inhibition of oligomer formation. The photo-induced cross-linking of unmodified proteins (PICUP) assay was used to investigate α-syn (60 μM) and tau isoform 2N4R (6 μM) oligomerization in the presence of eight amide compounds (2, 4, 5, 6, 7, 8, 13, and 17 at 50 μM, i.e., ~molar ratio 1:8 for tau). After being subjected to short light exposure, α-syn and tau were cross-linked and Coomassie-stained resolved as a higher molecular weight compared to control (0.125% DMSO). Compounds 2, 8, and 17 effectively prevented this crosslinking for both α-syn and tau isoform 2N4R oligomerization. Compound 13 was used as a negative control for the α-syn PICUP. However, the compound resulting in the strongest oligomer band (i.e., compound 6) was selected as negative control for the tau PICUP and resulted in high molecular bands representing the oligomeric species. Compounds 2, 8, and 17 proved to be potent inhibitors of α-syn and tau aggregation by reducing not only fibrilization but also oligomerization.

One limitation of the PICUP is the generation of free radicals which can be quenched by aromatic compounds resulting in false positives. For this reason, results obtained by PICUP were validated using the uncross-linked α-syn. High concentration of different compounds (600 μM; molar ratio of 1:10) were incubated at 37° C. for 24 hours with 60 μM of α-syn and separated by electrophoresis in 16% SDS-PAGE gels. Western blots using polyclonal anti-oligo A11 and monoclonal anti-α-syn were assessed to detect the high molecular weight oligomers and monomeric α-syn, respectively. The anti-oligomeric effect of 4-aminoindole carboxamide derivatives 2 and 8 were confirmed by narrow, less intense bands compared to control (1.5% DMSO). Compounds 13 (negative control) and 17 (anti-fibrillary compound) did not reduce the intensity of the bands representing the oligomeric species.

### Example 5

### Dose-Response Analysis

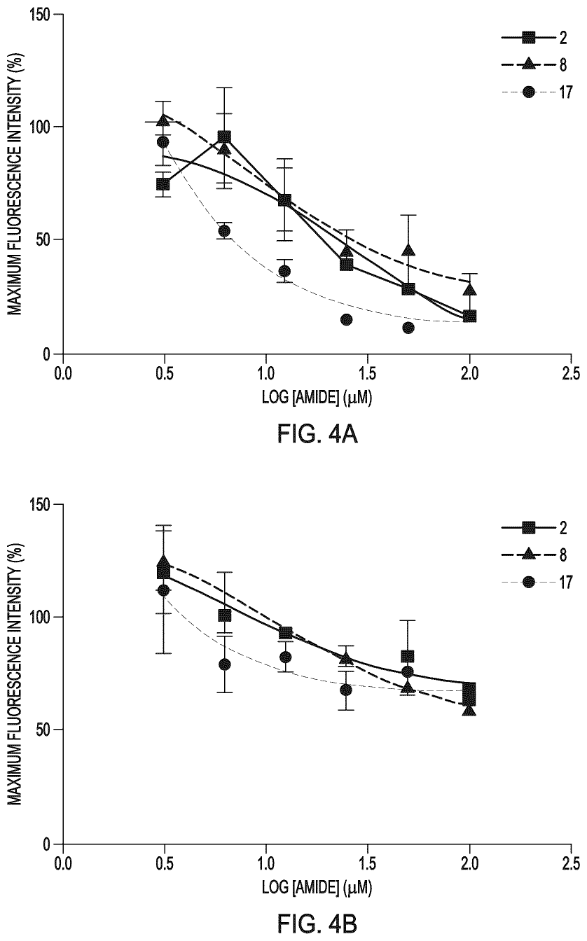

The three lead compounds (2, 8, and 17) were tested at a lower molar ratio to analyze their effects on α-syn and tau aggregation kinetics and demonstrated a dose-dependent relationship between concentration of compound and aggregation of protein. For each concentration (3.125, 6.25, 12.5, 25, 50 and 100 μM), triplicate data were collected from the five consecutive time points at the plateau phase. α-Syn was tested at 2 μM. For tau (used at 6 μM), the resulting molar ratios (protein:compound) consist of ~1:0 (control DMSO), 1:0.5 (compound at 3.125 μM), 1:1 (compound at 6.25 μM), 1:2 (compound at 12.5 μM), 1:4 (compound at 25 μM), 1:8 (compound at 50 μM), and 1:16 (compound at 100 μM). Dose-curve response was assessed with all three compounds. The data (FIGS. 4A-4B) showed a correlation between the concentration of the three lead compounds (dose-response) and a reduction in fluorescence intensity. Both α-syn and tau isoform 2N4R exhibited a reduction in protein fibrillization when subjected to higher concentrations of the three lead compounds.

PICUP assays were performed with different concentrations (50 μM, 25 μM, 12.5 μM, 6.25 μM, 3.125 μM; control=0.125% DMSO) for each lead compound. PICUP dose-response experiments further confirmed that compounds 2, 8, and 17 prevented α-syn oligomer formation in a dose-dependent manner. Compounds 2, 8, and 17 prevented tau 2N4R oligomer formation at a concentration of 50 μM.

### Example 6

Transmission electron microscopy analysis of anti-fibrillar activity of compounds on α-syn and tau isoform 2N4R

For the examination of ultrastructural changes, 60 μM of proteins (α-syn and tau isoform 2N4R) in contact with DMSO or 600 μM of compound were incubated in a 10 mM PBS buffer (pH 7.4) for 24 h at 37° C. (molar ratio 1:10). Samples at the end of tau ThT kinetics of fibril formation were also analyzed at similar molar ratio with 6 time less concentrated samples. For the preparation of grids prior examination, a volume of 10 μL if each sample was applied on a 400-mesh Formvar-carbon-coated copper grid (Electron Microscopy Sciences, Hatfield, PA). The grids were incubated for 1 minute with the sample and after washed three times with distilled water, After being air-dried, a fresh solution of 1% uranyl acetate was applied for 1 minute. Solution was absorbed with filter paper and grids were air-dried. Evaluation of grids were performed using a transmission microscope (JEOL 1400 Flash, Japan). Transmission electron micrographs were captured using an accelerating voltage of 100 kV and magnification of 40 k.

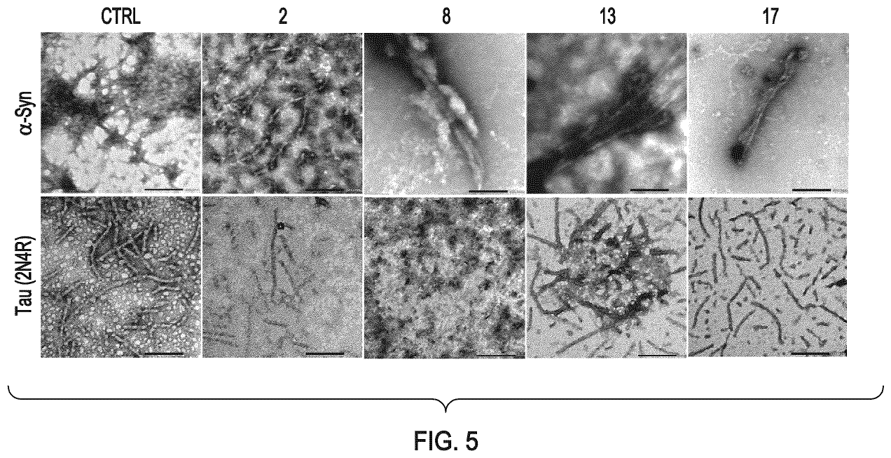

To confirm that the reduction of ThT fluorescence observed with compounds 2, 8, and 17 was due to the reduction in protein fibrillization rather than interference with ThT [31], transmission electron microscopy (TEM) of α-syn and tau isoform 2N4R with these same compounds was utilized to visualize direct changes in α-syn and tau isoform 2N4R fibril morphology after 24-hours exposure (FIG. 5). All three compounds exhibited a clear effect in reducing α-syn fibrillization at a molar ratio 1:10 (60 μM α-syn: 600 μM compound or 1.5% DMSO). Treatment of α-syn with compound 8 resulted in the most drastic effect, with immature fibrils surrounded by irregular globular structures. Dense mats of fibrils were observed with the DMSO control but not under compounds 2, 8, and 17. Compound 13 was used as a control and treated neuropeptides resulted in mature fibrils after 24 hours. For the tau isoform 2N4R the levels of tau fibril at 20 μM were reduced after 50-hours exposure with compounds 2 and 8 at 100 μM (molar ratio 1:10; control=0.25% DMSO) (FIG. 5). Results were reproduced using the tau isoform 2N4R and different compounds at the above-mentioned higher concentrations using the identical molar ratio and incubation time. Long fibrils were observed with DMSO control (CTRL) and compound 17. Fewer and not well-defined fibrils were observed on the grid prepared with compound 2 treated sample. Compound 2 was the only candidate resulting in a grid quasi exempt of fibrils. Compound 13 exhibited long fibrils admixed with amorphous materials and/or debris. Hence, compounds 2 and 8 were identified as the best compounds to reduce α-syn and tau isoform 2N4R fibril formation.

### Example 7

### Evaluation of Compounds Against α-Syn Inclusion in Neuroblastoma Cells

Dox-inducible neuroblastoma cells M17D-TR/αS-3K::YFP were used according to the published experimental procedure [26, 27, 32]. For this cell-based assay, the cellular density consisted of 30,000 cells per well in a 96-well plate format. After 24 h of seeding the plates, compounds were introduced at ranging concentrations of 5 to 40 μM for a period of 24 h. After, the induction of the αS-3K::YFP transgene expression was initiated by adding dox (1 ug per mL, final concentration in culture media). Cells were incubated in the Incucyte Zoom 2000 platform (Essen Biosciences), where the acquisition of images (green, bright field) occurred continuously. After 48 hours of induction, inclusion formation and cell growth were assessed. To determine the level of different proteins by western blotting, α-syn monoclonal antibody 41312 (Thermofisher, Waltham, MA; 1:1000) and GAPDH polyclonal antibody (Sigma-Aldrich, St. Louis, MO, G9545; 1:5000) were used.

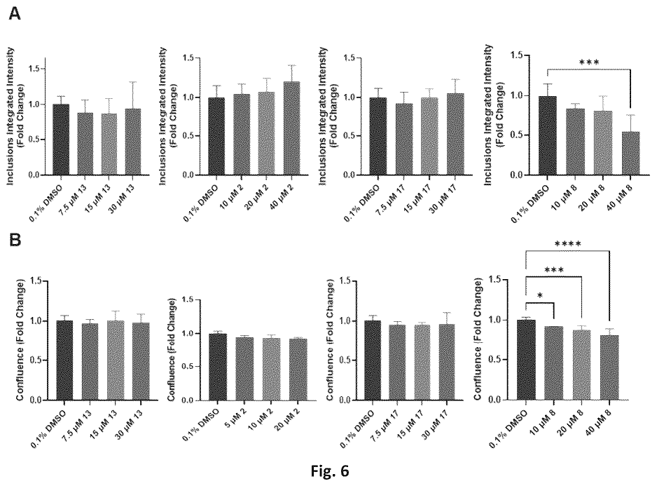

To evaluate the effects of compounds on inclusion formation and cell survival, M17D neuroblastoma cells that express an α-syn-derived αS-3K::YFP fusion protein in a doxycycline-inducible fashion were used. The cells were treated with 0.1% DMSO (control) and compounds 13, 2, 8, and 17 at various concentrations at t=24 hrs. Cells were induced with dox at t=48 hours. Punctate YFP signals were measured and normalized to 0.1% DMSO at t=96 hours. Eight independent experiments (N=8; n=14; except DMSO, n=30) were performed. The familial PD-linked α-syn missense mutation E46K is amplified by α-syn E35K+E46K+E61K (=αS3K), which results in round cytoplasmic α-syn-based inclusions in cultured cells. αS3K expression is responsible for cell stress/toxicity and delayed growth of neuroblastoma cells. In the αS3K system, several sulfonamide, urea, and other derivative compounds have been demonstrated to overcome both αS inclusion formation and αS-induced cytotoxicity. Therefore, this assay was selected to evaluate the effect of the compound 13, as a weak to non-inhibitor of aggregation, and the three best anti-aggregation inhibitors, namely compounds 2, 8, and 17 (FIG. 6). Compound 8 reduced inclusion formation in M17D αS3K neuroblastoma cells at a concentration of 40 μM (FIG. 6B, left). None of these compounds exhibited a significant reduction of inclusions at lower concentrations. Only compound 8 exhibited a small degree of toxicity at 40 μM (FIG. 6B, right). Western blot analyses for total α-syn, normalized to calnexin (loading control), were performed to determine the compound effect on α-syn protein level due to degradation or non-specific effect on transgene expression. Compound 8 reduced α-syn levels at low concentration (5-10 μM) only.

TABLE 1

| | | | | | | | | | | | | α-Syn |
|---|---|---|---|---|---|---|---|---|---|---|---|---|
| ID# | Structure | MW | TPSA | LogP | LogD | BBB | LR5 | pKa | HBD | HBA | MPO | ThT% |
| Control | | | | | | | | | | | | 100 ± 6.3 |
| 1 | 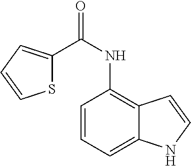 [SMILES: O=C(Nc1cccc2[nH]ccc12)c1cccs1] | 242.2963 | 73.13 | 2.77 | 3.08 | Y | Y | 14.3 | 2 | 1 | 2.6 | 63.5 ± 23.6 |
| 2 | 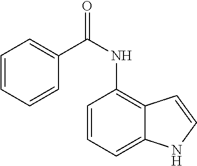 [SMILES: O=C(Nc1cccc2[nH]ccc12)c1ccccc1] | 236.2686 | 44.89 | 2.72 | 3.16 | Y | Y | 15.63 | 2 | 1 | 3.92 | 27.3 ± 0.5 |
| 3 | 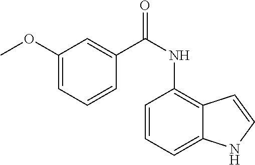 [SMILES: COc1cccc(C(=O)Nc2cccc3[nH]ccc23)c1] | 266.2946 | 54.12 | 2.74 | 3.01 | Y | Y | 15.06 | 2 | 2 | 3.9 | 42.8 ± 11.6 |
| 4 | 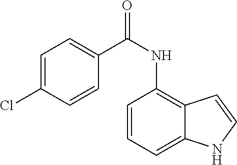 [SMILES: O=C(Nc1cccc2[nH]ccc12)c1ccc(Cl)cc1] | 270.7136 | 44.89 | 3.26 | 3.77 | Y | Y | 15.07 | 2 | 1 | 3.5 | 22.0 ± 0.2 |
| 5 | 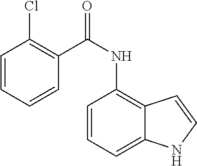 [SMILES: O=C(Nc1cccc2[nH]ccc12)c1ccccc1Cl] | 270.7136 | 44.89 | 3.25 | 3.77 | Y | Y | 14.92 | 2 | 1 | 3.49 | 19.5 ± 1.8 |
| 6 | 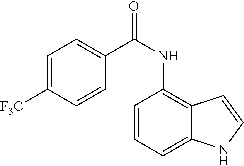 [SMILES: O=C(Nc1cccc2[nH]ccc12)c1ccc(C(F)(F)F)cc1] | 304.2665 | 44.89 | 3.77 | 4.04 | Y | Y | 15.05 | 2 | 1 | 3.1 | 26.7 ± 1.3 |
| 7 | 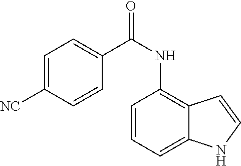 [SMILES: N#Cc1ccc(C(=O)Nc2cccc3[nH]ccc23)cc1] | 261.278 | 68.68 | 2.50 | 3.02 | Y | Y | 14.86 | 2 | 2 | 4.0 | 22.9 ± 2.7 |
| 8 | 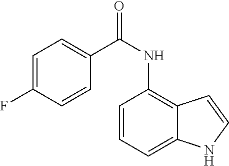 [SMILES: O=C(Nc1cccc2[nH]ccc12)c1ccc(F)cc1] | 254.259 | 65.12 | 3.03 | 3.31 | Y | Y | 14.80 | 2 | 1 | 3.83 | 22.2 ± 3.5 |
| 9 | 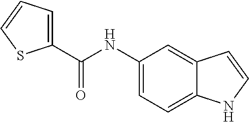 [SMILES: O=C(Nc1ccc2[nH]ccc2c1)c1cccs1] | 242.2963 | 73.13 | 2.80 | 3.08 | Y | Y | 14.34 | 2 | 1 | 4.0 | 40.5 ± 2.3 |
| 10 | 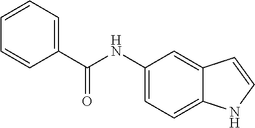 [SMILES: O=C(Nc1ccc2[nH]ccc2c1)c1ccccc1] | 236.2686 | 44.89 | 2.58 | 3.16 | Y | Y | 15.67 | 12 | 1 | 3.9 | 102.3 ± 13.3 |
| 11 | 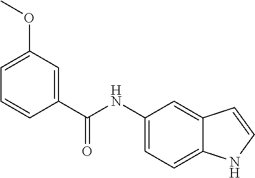 [SMILES: COc1cccc(C(=O)Nc2ccc3[nH]ccc3c2)c1] | 266.2946 | 54.12 | 2.72 | 3.01 | Y | Y | 15.10 | \| 2 | 2 | 3.9 | 48.6 ± 2.6 |
| 12 | 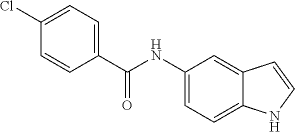 [SMILES: O=C(Nc1ccc2[nH]ccc2c1)c1ccc(Cl)cc1] | 270.7136 | 44.89 | 3.11 | 3.77 | Y | Y | 15.11 | 2 | 1 | 3.6 | 67.5 ± 16.3 |
| 13 | 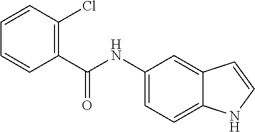 [SMILES: O=C(Nc1ccc2[nH]ccc2c1)c1ccccc1Cl] | 270.7136 | 44.89 | 3.24 | 3.77 | Y | Y | 14.96 | 2 | 1 | 3.5 | 73.7 ± 5.6 |
| 14 | 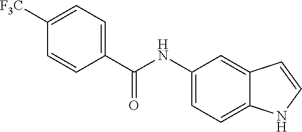 [SMILES: O=C(Nc1ccc2[nH]ccc2c1)c1ccc(C(F)(F)F)cc1] | 304.2665 | 44.89 | 3.75 | 4.04 | Y | Y | 15.09 | 2 | 1 | 3.1 | 64.4 ± 1.6 |
| 15 | 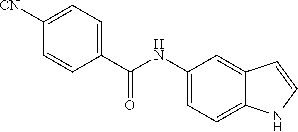 | 261.278 | 68.68 | 2.47 | 3.02 | Y | Y | 14.90 | 2 | 2 | 3.9 | 62.7 ± 4.1 |
| 16 | 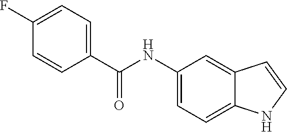 [SMILES: O=C(Nc1ccc2[nH]ccc2c1)c1ccc(F)cc1] | 254.259 | 44.89 | 2.89 | 3.31 | Y | Y | 14.84 | 2 | 1 | 3.8 | 48.5 ± 1.7 |
| 17 | 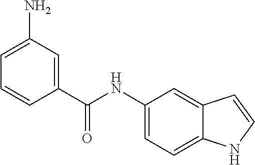 [SMILES: Nc1cccc(C(=O)Nc2ccc3[nH]ccc3c2)c1] | 251.280 | 70.91 | 2.03 | 2.33 | Y | Y | 15.70 | 3 | 2 | 4.0 | 44.1 ± 11.9 |
| 18 | 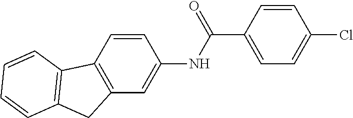 [SMILES: O=C(Nc1ccc2c(c1)Cc1ccccc1-2)c1ccc(Cl)cc1] | 251.2832 | 29.10 | 4.34 | 4.95 | Y | N | 14.57 | 1 | 1 | 2.6 | 69.4 ± 6.70 |

Seventeen aminoindole carboxamides were designed and synthesized to investigate their potential therapeutic effects on α-syn and tau isoform 2N4R fibril formation. It was beneficial to perform the anti-aggregation studies on α-syn first as a pre-test to identify potential candidates to further evaluate their tau anti-fibrillization activities. The results obtained from the α-syn ThT fluorescence analysis demonstrated that the 4-aminoindole derivatives were more potent inhibitor of fibril formation in contrast to the 5-aminoindole derivatives. PICUP assay allowed us to identify compounds 2, 8, 17 as the best molecules for reducing oligomerization of α-syn. These three aminoindole carboxamides reduced the α-syn and tau isoform 2N4R fibril formation as well as the α-syn oligomerization in a dose-dependent manner. Compounds 2 and 8 are both representative of the 4-aminoindole series whereas compound 17 is a representative of the 5-aminoindole series. Compound 17 harbors a free amine that would need further optimization to avoid structure alert (i.e., oxidation by cytochrome p450 and formation of carcinogenic metabolites). All three compounds possess physicochemical properties favorable to cross the blood brain barrier. Moreover, compound 8 reduced α-syn inclusion body formation in the dox-inducible neuroblastoma cells M17D-TR/αS-3K::YFP at 40 μM. Based on the data obtained from the biological evaluation, compound 8 seems to be the most promising in preventing fibril formation, oligomerization, and cell inclusion.

The amide scaffold is of utmost interest in developing new anti-NFT therapies given their non-cytotoxic impact to various cell lines and their drug-like properties, including a high propensity to cross the BBB. The best anti-aggregation compounds with α-syn and inhibition of tau isoform 2N4R fibril formation were pre-selected using 4-aminoindole carboxamide derivative compounds. These amide derivatives could represent a new class of effective inhibitors of prone-to-aggregate proteins relevant for the development of new therapies for neurodegenerative diseases.

### Example 8

### Kinetics of Solubility

Compounds 2 and 8 (best compounds) and one negative control (compound 13) were solubilized in ThT buffer comprised of 1×PBS at pH=7.4 with additional 300 mM NaCl and 0.5 mM SDS. The anti-fibrillization effect observed with compounds 2 and 8 was independent of the solubility. Kinetics of solubility analysis was performed at the MSU Medicinal Chemistry Core facility.

TABLE 2
Kinetics of solubility

| Compound | Measured Solubility |
|---|---|
| 2 | >300 μM |
| 8 | >300 μM |
| 13 | >300 μM |
| Mebendazole | 30 μM |

### Example 9

The following additional compounds (Table 3) can be synthesized using methods similar to those described in Example 1.

TABLE 3

| | | |
|---|---|---|
| 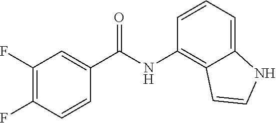 [SMILES: O=C(Nc1cccc2[nH]ccc12)c1ccc(F)c(F)c1] | 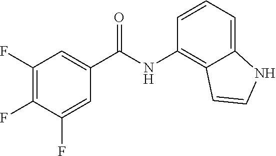 [SMILES: O=C(Nc1cccc2[nH]ccc12)c1cc(F)c(F)c(F)c1] | 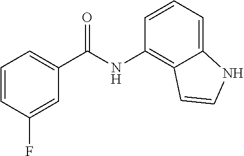 [SMILES: O=C(Nc1cccc2[nH]ccc12)c1cccc(F)c1] |
| 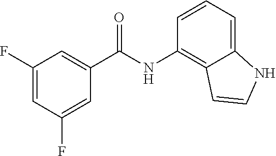 [SMILES: O=C(Nc1cccc2[nH]ccc12)c1cc(F)cc(F)c1] | 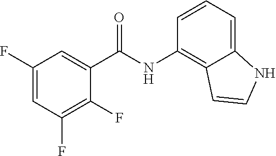 [SMILES: O=C(Nc1cccc2[nH]ccc12)c1cc(F)cc(F)c1F] | 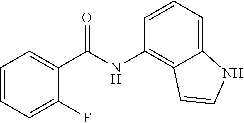 [SMILES: O=C(Nc1cccc2[nH]ccc12)c1ccccc1F] |
| 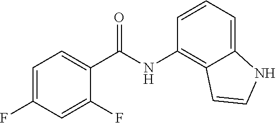 [SMILES: O=C(Nc1cccc2[nH]ccc12)c1ccc(F)cc1F] | 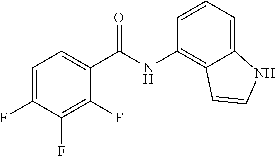 [SMILES: O=C(Nc1cccc2[nH]ccc12)c1ccc(F)c(F)c1F] | 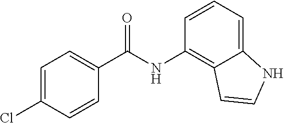 [SMILES: O=C(Nc1cccc2[nH]ccc12)c1ccc(Cl)cc1] |
| 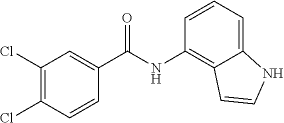 [SMILES: O=C(Nc1cccc2[nH]ccc12)c1ccc(Cl)c(Cl)c1] | 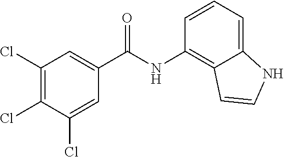 [SMILES: O=C(Nc1cccc2[nH]ccc12)c1cc(Cl)c(Cl)c(Cl)c1] | 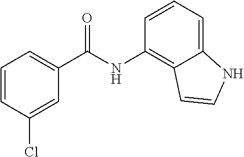 [SMILES: O=C(Nc1cccc2[nH]ccc12)c1cccc(Cl)c1] |
| 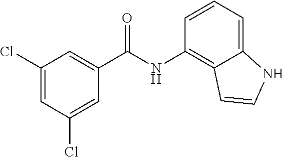 [SMILES: O=C(Nc1cccc2[nH]ccc12)c1cc(Cl)cc(Cl)c1] | 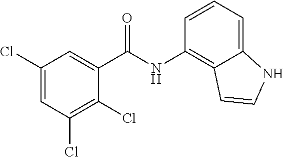 [SMILES: O=C(Nc1cccc2[nH]ccc12)c1cc(Cl)cc(Cl)c1Cl] | 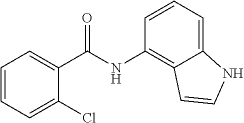 [SMILES: O=C(Nc1cccc2[nH]ccc12)c1ccccc1Cl] |
| 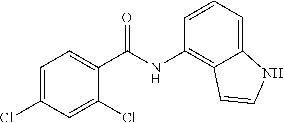 [SMILES: O=C(Nc1cccc2[nH]ccc12)c1ccc(Cl)cc1Cl] | 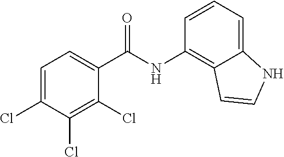 [SMILES: O=C(Nc1cccc2[nH]ccc12)c1ccc(Cl)c(Cl)c1Cl] | 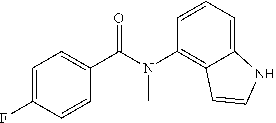 [SMILES: CN(C(=O)c1ccc(F)cc1)c1cccc2[nH]ccc12] |
| 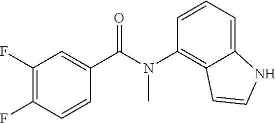 [SMILES: CN(C(=O)c1ccc(F)c(F)c1)c1cccc2[nH]ccc12] | 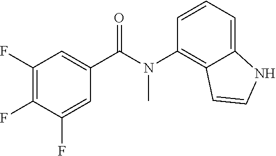 [SMILES: CN(C(=O)c1cc(F)c(F)c(F)c1)c1cccc2[nH]ccc12] | 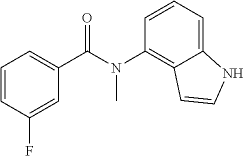 [SMILES: CN(C(=O)c1cccc(F)c1)c1cccc2[nH]ccc12] |
| 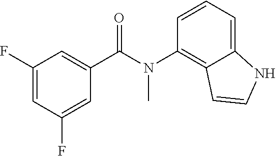 [SMILES: CN(C(=O)c1cc(F)cc(F)c1)c1cccc2[nH]ccc12] | 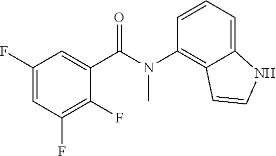 [SMILES: CN(C(=O)c1cc(F)cc(F)c1F)c1cccc2[nH]ccc12] | 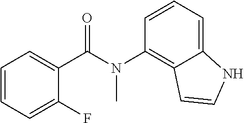 [SMILES: CN(C(=O)c1ccccc1F)c1cccc2[nH]ccc12] |
| 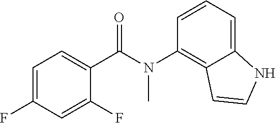 [SMILES: CN(C(=O)c1ccc(F)cc1F)c1cccc2[nH]ccc12] | 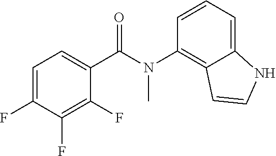 [SMILES: CN(C(=O)c1ccc(F)c(F)c1F)c1cccc2[nH]ccc12] | 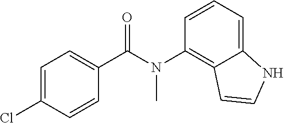 [SMILES: CN(C(=O)c1ccc(Cl)cc1)c1cccc2[nH]ccc12] |
| 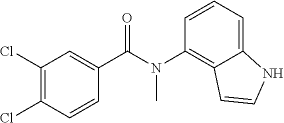 [SMILES: CN(C(=O)c1ccc(Cl)c(Cl)c1)c1cccc2[nH]ccc12] | 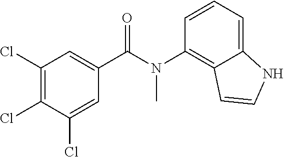 [SMILES: CN(C(=O)c1cc(Cl)c(Cl)c(Cl)c1)c1cccc2[nH]ccc12] | 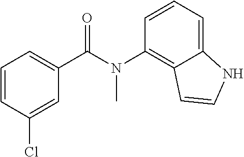 [SMILES: CN(C(=O)c1cccc(Cl)c1)c1cccc2[nH]ccc12] |
| 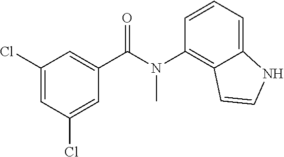 [SMILES: CN(C(=O)c1cc(Cl)cc(Cl)c1)c1cccc2[nH]ccc12] | 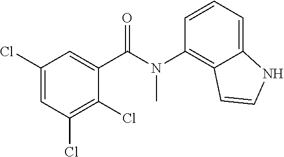 [SMILES: CN(C(=O)c1cc(Cl)cc(Cl)c1Cl)c1cccc2[nH]ccc12] | 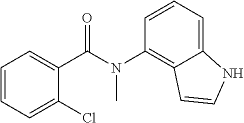 [SMILES: CN(C(=O)c1ccccc1Cl)c1cccc2[nH]ccc12] |
| 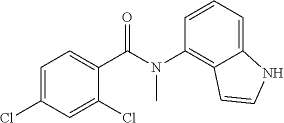 [SMILES: CN(C(=O)c1ccc(Cl)cc1Cl)c1cccc2[nH]ccc12] | 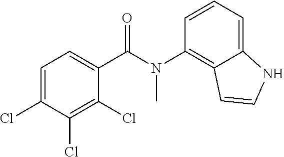 [SMILES: CN(C(=O)c1ccc(Cl)c(Cl)c1Cl)c1cccc2[nH]ccc12] | 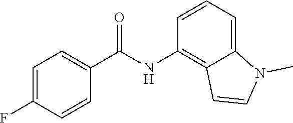 [SMILES: Cn1ccc2c(NC(=O)c3ccc(F)cc3)cccc21] |
| 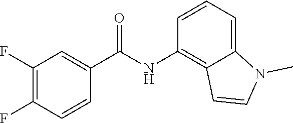 [SMILES: Cn1ccc2c(NC(=O)c3ccc(F)c(F)c3)cccc21] | 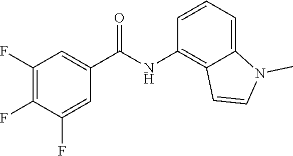 [SMILES: Cn1ccc2c(NC(=O)c3cc(F)c(F)c(F)c3)cccc21] | 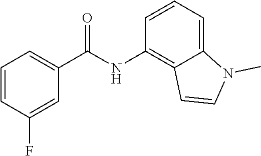 [SMILES: Cn1ccc2c(NC(=O)c3cccc(F)c3)cccc21] |
| 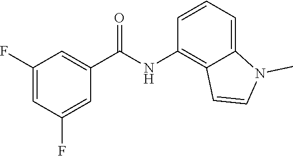 [SMILES: Cn1ccc2c(NC(=O)c3cc(F)cc(F)c3)cccc21] | 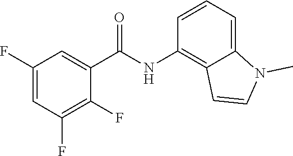 [SMILES: Cn1ccc2c(NC(=O)c3cc(F)cc(F)c3F)cccc21] | 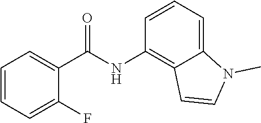 [SMILES: Cn1ccc2c(NC(=O)c3ccccc3F)cccc21] |
| 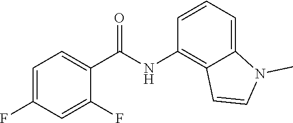 [SMILES: Cn1ccc2c(NC(=O)c3ccc(F)cc3F)cccc21] | 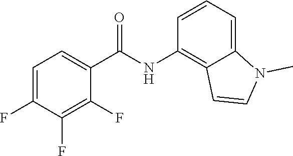 [SMILES: Cn1ccc2c(NC(=O)c3ccc(F)c(F)c3F)cccc21] | 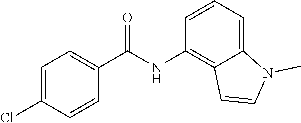 [SMILES: Cn1ccc2c(NC(=O)c3ccc(Cl)cc3)cccc21] |
| 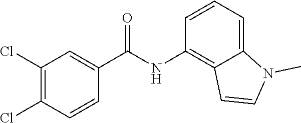 [SMILES: Cn1ccc2c(NC(=O)c3ccc(Cl)c(Cl)c3)cccc21] | 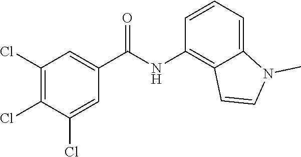 [SMILES: Cn1ccc2c(NC(=O)c3cc(Cl)c(Cl)c(Cl)c3)cccc21] | 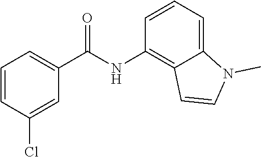 [SMILES: Cn1ccc2c(NC(=O)c3cccc(Cl)c3)cccc21] |
| 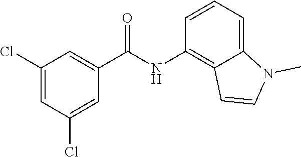 [SMILES: Cn1ccc2c(NC(=O)c3cc(Cl)cc(Cl)c3)cccc21] | 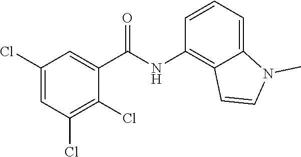 [SMILES: Cn1ccc2c(NC(=O)c3cc(Cl)cc(Cl)c3Cl)cccc21] | 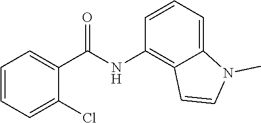 [SMILES: Cn1ccc2c(NC(=O)c3ccccc3Cl)cccc21] |
| 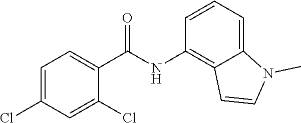 [SMILES: Cn1ccc2c(NC(=O)c3ccc(Cl)cc3Cl)cccc21] | 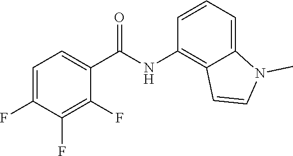 [SMILES: Cn1ccc2c(NC(=O)c3ccc(F)c(F)c3F)cccc21] | 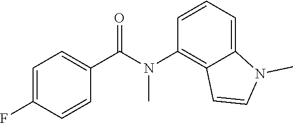 [SMILES: CN(C(=O)c1ccc(F)cc1)c1cccc2c1ccn2C] |
| 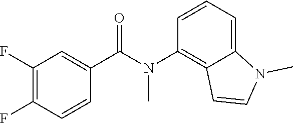 [SMILES: CN(C(=O)c1ccc(F)c(F)c1)c1cccc2c1ccn2C] | 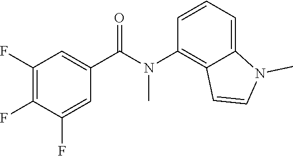 [SMILES: CN(C(=O)c1cc(F)c(F)c(F)c1)c1cccc2c1ccn2C] | 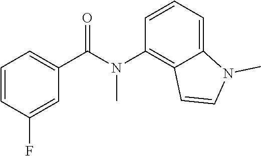 [SMILES: CN(C(=O)c1cccc(F)c1)c1cccc2c1ccn2C] |
| 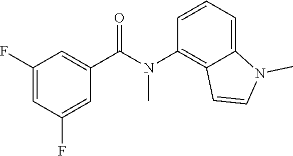 [SMILES: CN(C(=O)c1cc(F)cc(F)c1)c1cccc2c1ccn2C] | 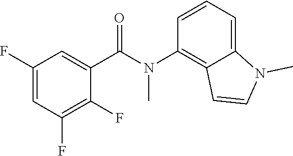 [SMILES: CN(C(=O)c1cc(F)cc(F)c1F)c1cccc2c1ccn2C] | 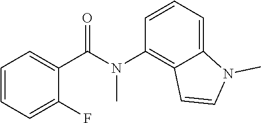 [SMILES: CN(C(=O)c1ccccc1F)c1cccc2c1ccn2C] |
| 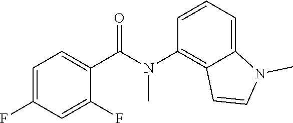 [SMILES: CN(C(=O)c1ccc(F)cc1F)c1cccc2c1ccn2C] | 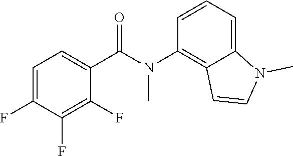 [SMILES: CN(C(=O)c1ccc(F)c(F)c1F)c1cccc2c1ccn2C] | 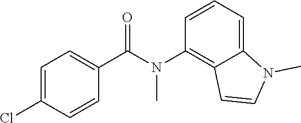 [SMILES: CN(C(=O)c1ccc(Cl)cc1)c1cccc2c1ccn2C] |
| 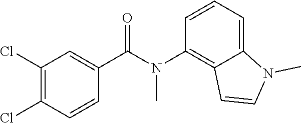 [SMILES: CN(C(=O)c1ccc(Cl)c(Cl)c1)c1cccc2c1ccn2C] | 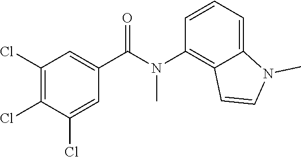 [SMILES: CN(C(=O)c1cc(Cl)c(Cl)c(Cl)c1)c1cccc2c1ccn2C] | 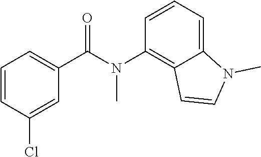 [SMILES: CN(C(=O)c1cccc(Cl)c1)c1cccc2c1ccn2C] |
| 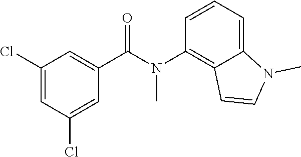 [SMILES: CN(C(=O)c1cc(Cl)cc(Cl)c1)c1cccc2c1ccn2C] | 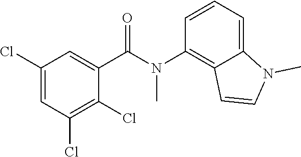 [SMILES: CN(C(=O)c1cc(Cl)cc(Cl)c1Cl)c1cccc2c1ccn2C] | 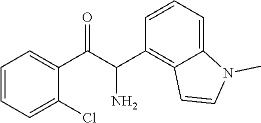 [SMILES: Cn1ccc2c(C(N)C(=O)c3ccccc3Cl)cccc21] |
| 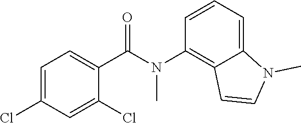 [SMILES: CN(C(=O)c1ccc(Cl)cc1Cl)c1cccc2c1ccn2C] | 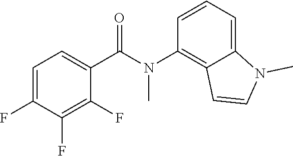 [SMILES: CN(C(=O)c1ccc(F)c(F)c1F)c1cccc2c1ccn2C] | |

### Example 10

For compound 8, the brain concentration and plasma concentration ratio is equal to more than 1 for the 3 time points (0.08, 0.5 and 1 hour) where the compound was detected, which is adequate. The compound was not detected beyond the time point of 60 min. Half-life is presumed to be short, and this could be explained by small quantity administered (1 mg/kg) and/or elimination, Mouse microsomal stability of the same compound resulted in 70% which is good.

All patents, patent application publications, journal articles, textbooks, and other publications mentioned in the specification are indicative of the level of skill of those in the art to which the disclosure pertains. All such publications are incorporated herein by reference to the same extent as if each individual publication were specifically and individually indicated to be incorporated by reference.

The invention illustratively described herein may be suitably practiced in the absence of any element(s) or limitation(s), which is/are not specifically disclosed herein. Thus, for example, each instance herein of any of the terms “comprising,” “consisting essentially of,” and “consisting of” may be replaced with either of the other two terms. Likewise, the singular forms “a,” “an,” and “the” include plural references unless the context clearly dictates otherwise. Thus, for example, references to “the method” includes one or more methods and/or steps of the type, which are described herein and/or which will become apparent to those ordinarily skilled in the art upon reading the disclosure.

The term “about,” when referring to a number or a numerical value or range (including, for example, whole numbers, fractions, and percentages), means that the number or numerical range referred to is an approximation within experimental variability (or within statistical experimental error) and thus the numerical value or range can vary between 1% and 15% of the stated number or numerical range (e.g., +/−5% to 15% of the recited value) provided that one of ordinary skill in the art would consider equivalent to the recited value (e.g., having the same function or result). The term “substantially” can allow for a degree of variability in a value or range, for example, within 90%, within 95%, or within 99% of a stated value or of a stated limit of a range.

The terms and expressions, which have been employed, are used as terms of description and not of limitation. In this regard, where certain terms are defined under “Definitions” and are otherwise defined, described, or discussed elsewhere in the “Detailed Description,” all such definitions, descriptions, and discussions are intended to be attributed to such terms. There also is no intention in the use of such terms and expressions of excluding any equivalents of the features shown and described or portions thereof. Furthermore, while subheadings, e.g., “Definitions,” are used in the “Detailed Description,” such use is solely for ease of reference and is not intended to limit any disclosure made in one section to that section only; rather, any disclosure made under one subheading is intended to constitute a disclosure under each and every other subheading.

It is recognized that various modifications are possible within the scope of the claimed invention. Thus, although the present invention has been specifically disclosed in the context of preferred embodiments and optional features, those skilled in the art may resort to modifications and variations of the concepts disclosed herein. Such modifications and variations are considered within the scope of the invention as claimed herein.

1. A compound of the formula: 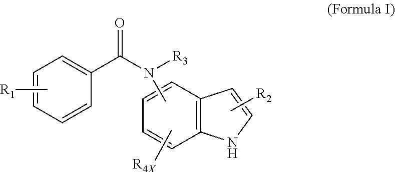
or a pharmaceutically acceptable salt thereof,
wherein R₁ is H, R^{a}O—, N≡C—, NO₂, NH₂, X₃C— or X, Rₐ is C₁-C₆ alkyl, and X is a halo;
R₂ is C₁-C₆ alkyl or X, wherein X is a halo;
R₃ is H or C₁-C₆ alkyl or X, wherein X is a halo; and
R₄ₓ, in which x is 1, 2 or 3, each R₄ is independently H, C₁-C₆ alkyl, or X, wherein X is a halo.

2. (canceled)

3. The compound of claim 1, having the structure: 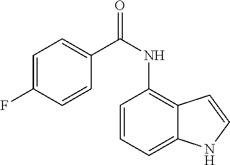 [SMILES: O=C(Nc1cccc2[nH]ccc12)c1ccc(F)cc1]
or a pharmaceutically acceptable salt thereof.

4. A compound of the formula: 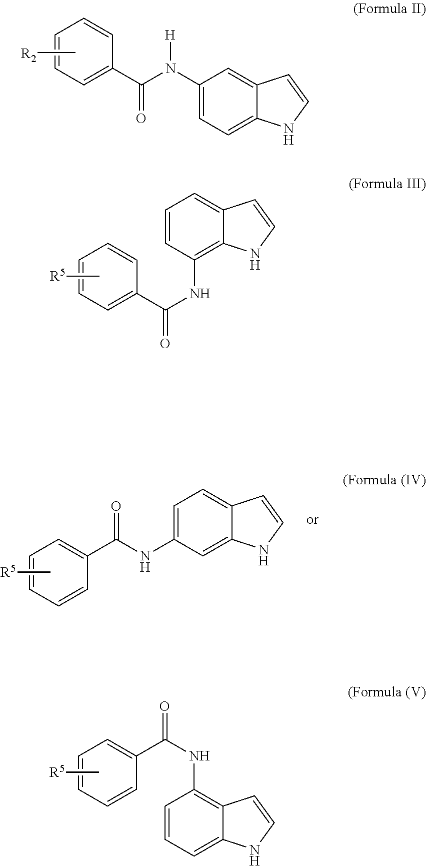
or a pharmaceutically acceptable salt thereof
wherein R₂ is H, R_{b}O—, N≡C—, NO₂, NH₂, X₃C— or X,
R^{b} is a C₁-C₆ alkyl, and
X is a halo.

5. (canceled)

6. A compound of the formula: 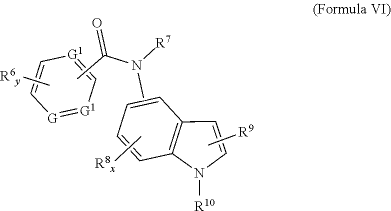
or a pharmaceutically acceptable salt thereof,
wherein:
G is N, CR⁶ or absent;
each G¹ is independently N, S, CR⁶;
each R⁶ is independently H, alkyl, alkoxy, aryl, aryloxy, alkoxy, N≡C—, NO₂, amino, X₃C— or halo or two R⁶ form a cycloalkyl group, an arylcycloalkyl group, an aryl group or a heterocyclyl group;
y is 1, 2, 3, or 4;
R⁷ is H or C₁-C₆ alkyl;
each R⁸ is C₁-C₆ alkyl or X, wherein X is a halo;
x is 1, 2 or 3; and
R⁹ is H, C₁-C₆ alkyl or X, wherein X is a halo.

7. The compound of claim 6, or a pharmaceutically acceptable salt thereof, wherein the compound is a compound of the formula:

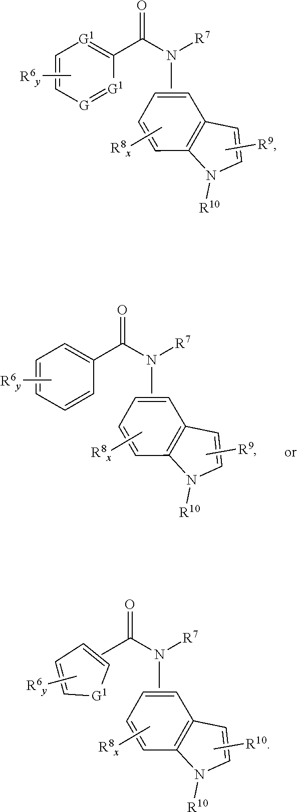

8. (canceled)

9. (canceled)

10. The compound of claim 6, or a pharmaceutically acceptable salt thereof, wherein the group of the formula: 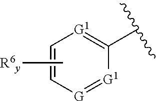
is a group of the formula:

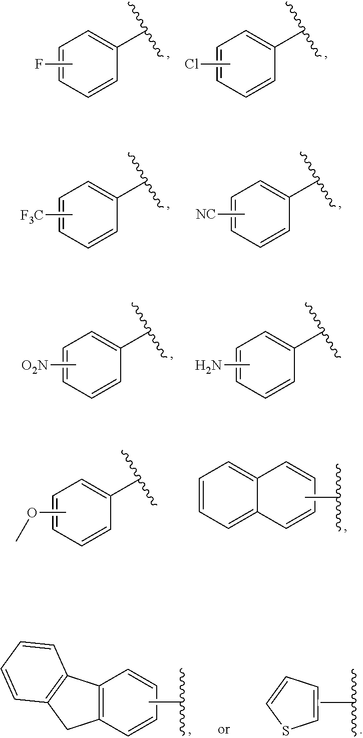

11. (canceled)

12. The compound of claim 6, or a pharmaceutically acceptable salt thereof, wherein R⁷ and/or R¹⁰ is C₁-C₃ alkyl.

13. (canceled)

14. (canceled)

15. (canceled)

16. The compound of claim 6, or a pharmaceutically acceptable salt thereof, wherein y is 2 or 3 and R⁶ is halo.

17. (canceled)

18. The compound of claim 10, or a pharmaceutically acceptable salt thereof, wherein y is 2 or 3 and R⁶ is halo.

19. (canceled)

20. The compound of claim 12, or a pharmaceutically acceptable salt thereof, wherein y is 2 or 3 and R⁶ is halo.

21. The compound of claim 6, or a pharmaceutically acceptable salt thereof, wherein y is 1 and R⁸ is H.

22. (canceled)

23. The compound of claim 10, or a pharmaceutically acceptable salt thereof, wherein y is 1 and R¹ is H.

24. (canceled)

25. The compound of claim 12, or a pharmaceutically acceptable salt thereof, wherein y is 1 and R¹ is H.

26. (canceled)

27. The compound of claim 6, or a pharmaceutically acceptable salt thereof, wherein R⁹ is H.

28. (canceled)

29. The compound of claim 10, or a pharmaceutically acceptable salt thereof, wherein R⁹ is H.

30. (canceled)

31. The compound of claim 12, or a pharmaceutically acceptable salt thereof, wherein R⁹ is H.

32. (canceled)

33. (canceled)

34. The compound of claim 1, wherein the compound is of the formula: 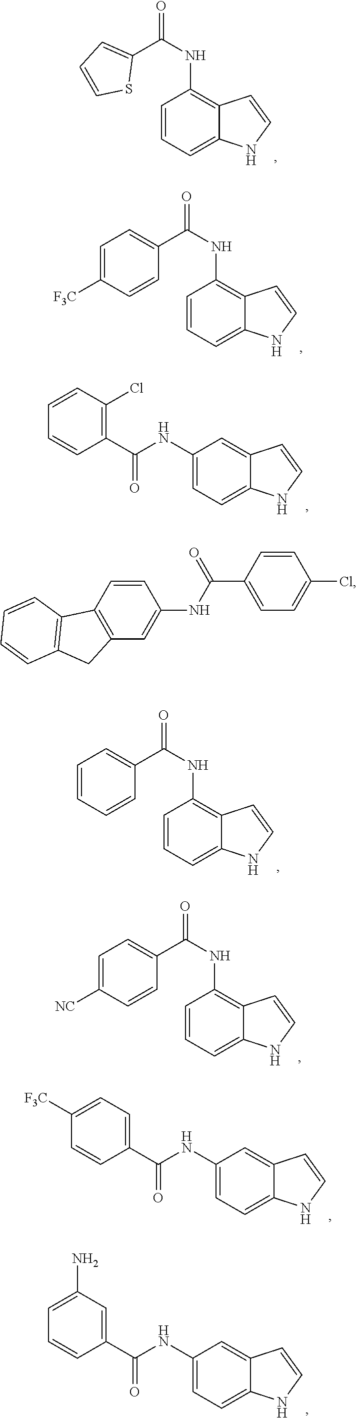 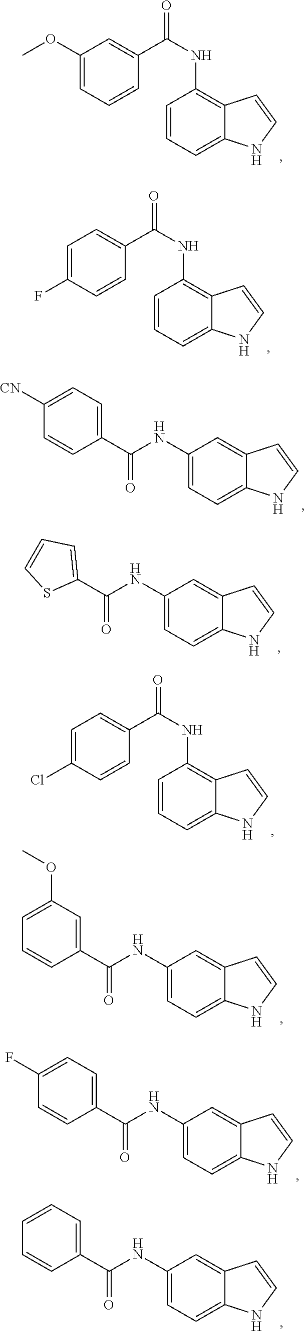 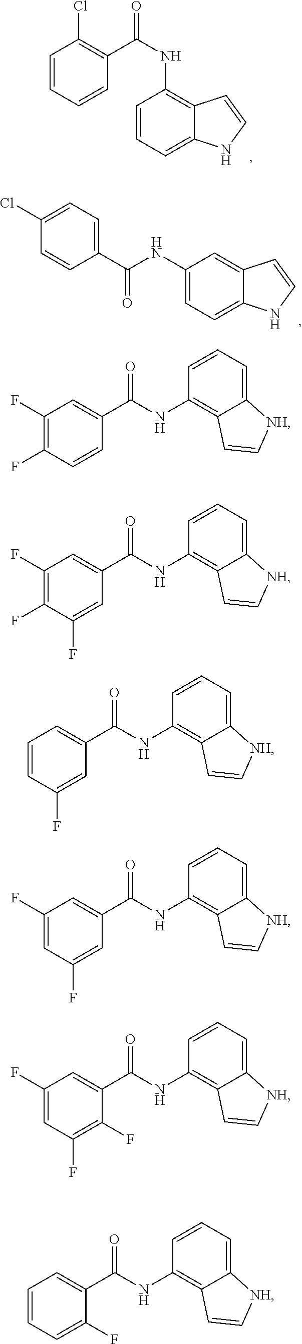 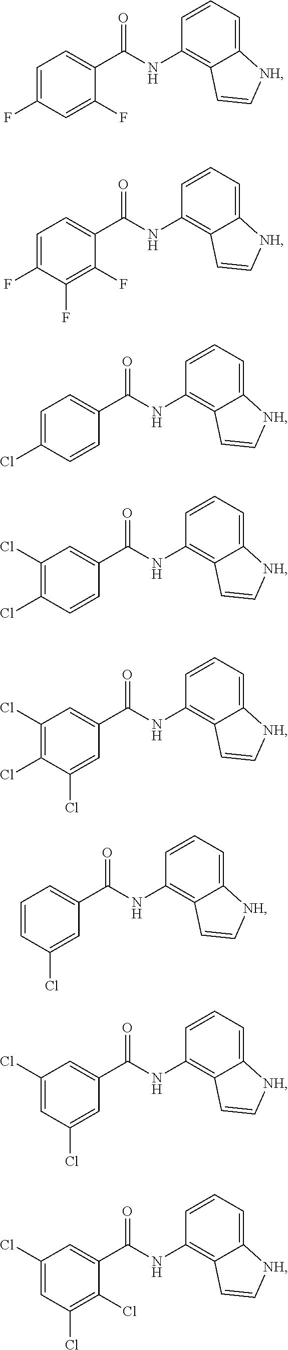 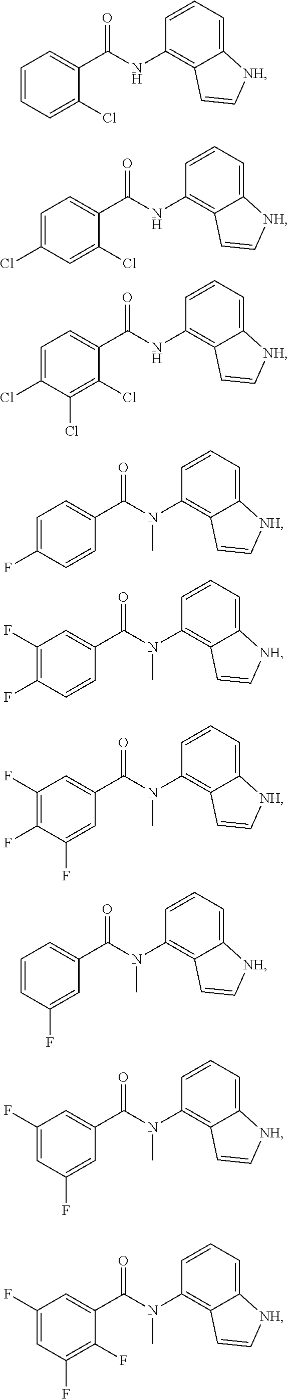 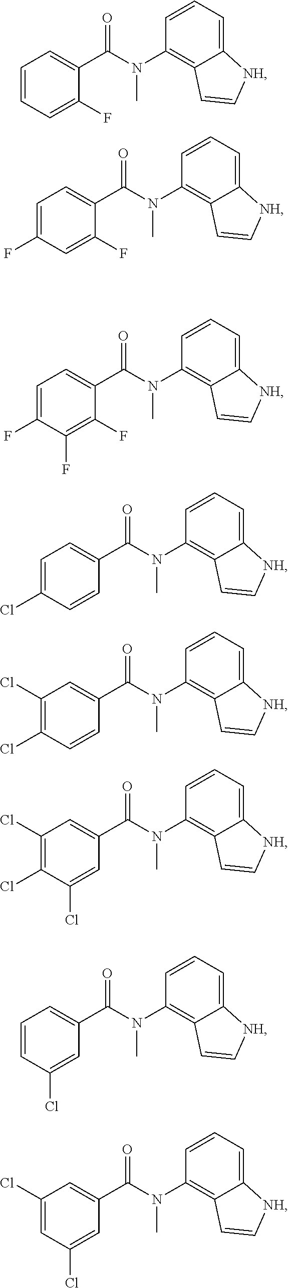 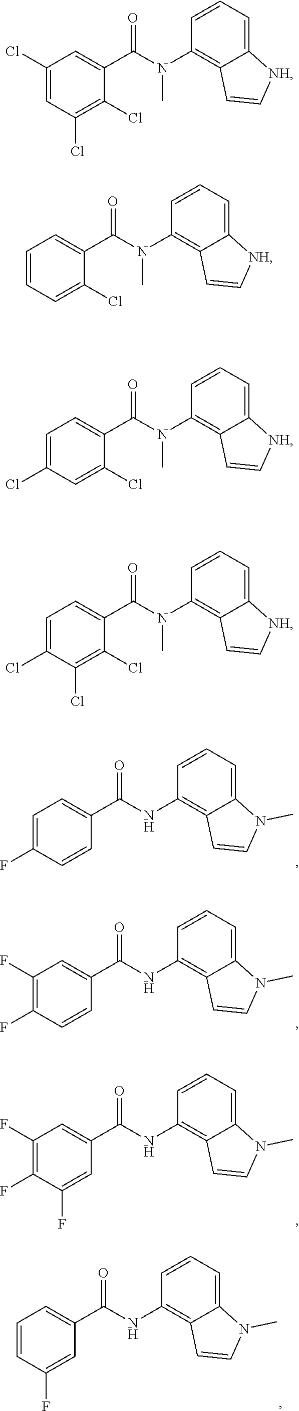 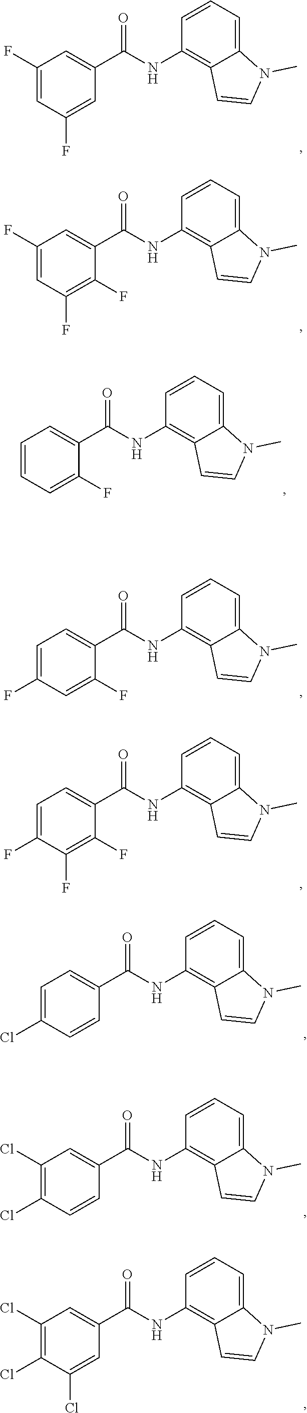 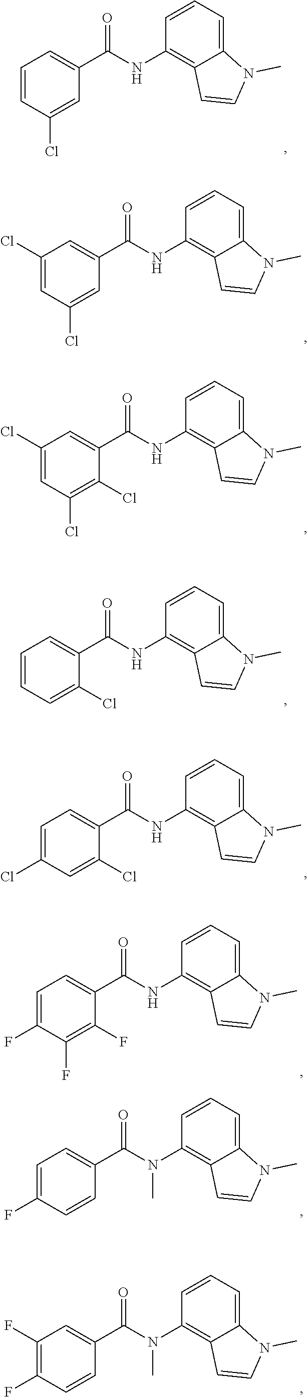 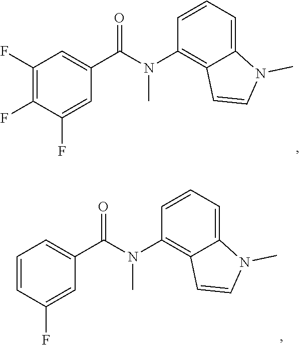 [SMILES: CN(C(=O)c1cc(F)c(F)c(F)c1)c1cccc2c1ccn2C ; CN(C(=O)c1cccc(F)c1)c1cccc2c1ccn2C] 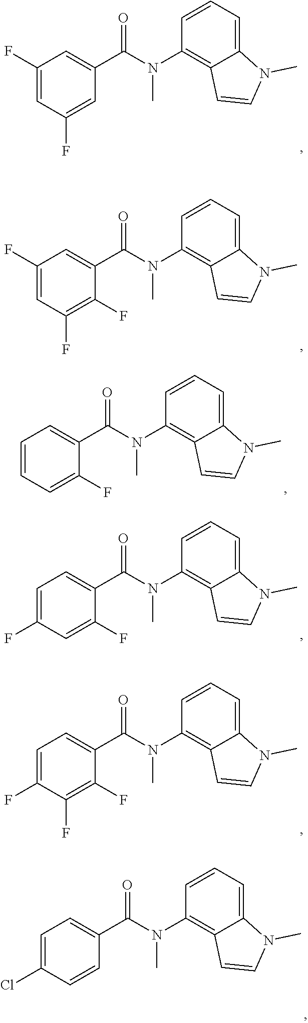 [SMILES: CN(C(=O)c1cc(F)cc(F)c1)c1cccc2c1ccn2C ; CN(C(=O)c1cc(F)cc(F)c1F)c1cccc2c1ccn2C ; CN(C(=O)c1ccccc1F)c1cccc2c1ccn2C ; CN(C(=O)c1ccc(F)cc1F)c1cccc2c1ccn2C ; CN(C(=O)c1ccc(F)c(F)c1F)c1cccc2c1ccn2C ; CN(C(=O)c1ccc(Cl)cc1)c1cccc2c1ccn2C] 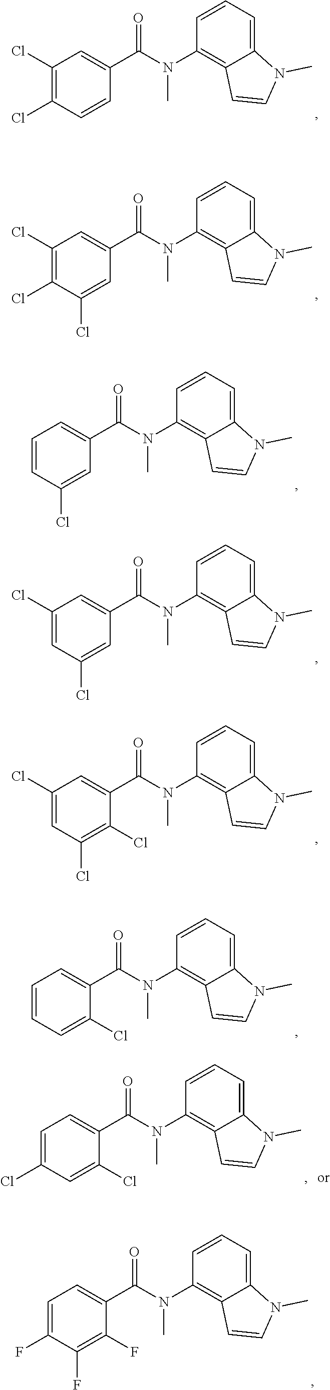
or a pharmaceutically acceptable salt thereof.

35. A pharmaceutical composition comprising the compound of claim 1, or a pharmaceutically acceptable salt thereof, and a pharmaceutically acceptable carrier.

36. A method of inhibiting tubulin-associated unit (tau) protein aggregation in a subject having, or at risk for, tau protein aggregation, which method comprises administering to the subject the composition of claim 35 in an amount effective to inhibit tau protein aggregation, whereupon tau protein aggregation is inhibited in the subject having, or at risk for, tau protein aggregation.

37. (canceled)

38. A method of inhibiting alpha-synuclein (α-syn) protein aggregation in a subject having, or at risk for, α-syn protein aggregation, which method comprises administering to the subject the composition of claim 35 in an amount effective to inhibit α-syn protein aggregation, whereupon α-syn aggregation is inhibited in the subject having, or at risk for, α-syn protein aggregation.

39. (canceled)
